(19) United States
(12) Patent Application Publication
(10) Pub. No.: US 2011/0218464 A1
(43) Pub. Date: Sep. 8, 2011

(54) System, Device and Methods of Tissue Treatment for Achieving Tissue Specific Effects

(75) Inventor: Yoni Iger, Haifa (IL)

(73) Assignee: Lumenis Ltd., Yokneam (IL)

(21) Appl. No.: 13/038,229

(22) Filed: Mar. 1, 2011

Related U.S. Application Data
(60) Provisional application No. 61/309,352, filed on Mar. 1, 2010.

(51) Int. Cl. A61B 18/18; A61N 7/00

(52) U.S. Cl. 601/2; 606/33

(57) ABSTRACT

A tissue treatment system and a treatment applicator provide multi-modality treatment of skin, tissue and organ conditions and pathologies using different types of treatment energy, alone or in combination, including radiofrequency (RF) energy and ultrasound energy to achieve multiple and different treatment effects that are tissue-specific and tissue depth-specific. The system and the applicator according to the invention selectively and fractionally and, as an option, nonfractionally, treat one or more tissue zones within a three-dimensional volume of skin or other soft tissue with one or more energy types and precisely target measured treatment energy to specific tissue types, tissue layers and/or specific depths or locations within the volume of skin or tissue. The system and the applicator can also target one or more energy types to treat a specific location or depth within a given tissue layer. The system and the applicator can provide different RF and ultrasound treatment energies that accurately affect the structure or activity of different tissue types, layers and/or depths within a given tissue zone of the skin or tissue volume in accordance with the treatment effects desired in particular tissue layers and/or at particular tissue depths to thereby selectively produce multiple and different treatment impacts.

### RELATED APPLICATIONS

This nonprovisional patent application claims priority to U.S. provisional patent application Ser. No. 61/309,352 filed Mar. 1, 2010, the disclosure of which is incorporated herein in its entirety by reference.

### FIELD OF THE INVENTION

A tissue treatment system, device and methods provide multi-modality treatment of skin and other soft tissue conditions and pathologies using different treatment energy modalities, alone or in combination, to achieve different and specific treatment effects within target tissue that relate to characteristics of target tissue including tissue type and tissue depth.

### BACKGROUND

Nonablative and ablative methods and techniques have been used in various dermatological, surgical, and other physical applications for treatment of conditions and pathologies of the human skin, tissues, and organs. Such methods and techniques employ different energy modalities, including radiofrequency energy and ultrasound energy to affect the structure and function of the skin, other soft tissues, and organs and to thereby therapeutically treat a particular condition and pathology. Various systems and techniques selectively deliver therapeutic energy to specific target tissues and organs in order the applied energy may have the intended therapeutic effect, while minimally affecting normal or surrounding tissues and organs. Such systems and techniques can apply treatment energy homogeneously over a treatment area, or, alternatively, can apply energy fractionally over a treatment area surface to focus and deliver energy in specific fractions along the X and Y axes of a given treatment area that leave portions of tissue within the area unaffected and intact. Intact tissue resulting from fractional treatments serves a number of purposes, including providing a blood supply to treated tissue to stimulate new cell production, a cellular reservoir to accelerate healing of purposefully damaged tissue, and a mechanical support for treated and untreated tissue within the treatment area.

Many skin, tissue and organ conditions and pathologies are best treated by causing specific and different physiological responses within a given treated tissue zone that result from the different type of tissue, layer of tissue and/or depth of tissue within the zone that receives therapeutic energy. For example, laser light is used in ablative and nonablative fractional skin resurfacing techniques that deliver laser light to a target tissue zone to selectively cause specific thermal responses within the zone, such as cell stimulation, blood vessel coagulation and tissue ablation, depending on the tissue type and/or depth within the zone that receives laser light treatment.

Use of combinations of different therapeutic energy modalities, including combinations of RF energy and/or ultrasound energy enhance the ability to selectively achieve specific and different physiological responses in a target tissue zone. Configurations and operation of such systems and techniques, however, can be improved, such that, different and multiple treatment responses and effects may be more precisely controlled and predicted and achieve improved volumetric impact in a given volume of target tissue. It is also desirable that such systems and techniques are configured to precisely treat different tissue layers located at different tissue depths according to the treatment impact desired or required, such as fractional and nonfractional impact, within a given volume of target tissue that are specific to the type, layer and/or depth of tissue treated. It is also desirable that such systems and techniques configure and deliver treatment energy to a given sub-volume of target tissue in a manner that controls the distribution and depth of energy induced heating in order different targeted tissue types, layers and depths are selectively treated and predicable three-dimensional heating profiles within the volume of target tissue may be reliably created.

### SUMMARY OF THE INVENTION

Generally, in one aspect, the invention provides a system for fractional treatment of a condition or pathology of a tissue including a treatment applicator disposed and configured to deliver treatment energy to a target tissue and defining within its interior a treatment cavity to engage the target tissue. The treatment applicator includes multiple bipolar RF electrodes disposed along predetermined internal surfaces that define the treatment cavity. Each bipolar RF electrode is disposed at an angle relative to the treatment cavity and is configured to generate RF energy. The multiple bipolar RF electrodes include a first set of at least two bipolar RF electrodes disposed along at least two internal surfaces of the treatment cavity. The at least two bipolar RF electrodes are electronically coupled to operate in a bipolar modality to deliver RF energy to the treatment cavity in a specific direction and at a specific angle so that the bipolar RF electrodes selectively target RF energy to at least one of: a specific layer, a specific depth, and/or a specific location or depth within a specific layer of a first zone within the target tissue. The first set of bipolar RF electrodes targets RF energy configured in accordance with one or more parameters to treat the first zone of the target tissue. The system further includes an RF energy source operatively coupled to the multiple bipolar RF electrodes.

Implementations of the invention may include one or more of the following features. The system includes a second set of at least two bipolar RF electrodes disposed along at least two internal surfaces of the treatment cavity. The at least two bipolar RF electrodes of the second set are electronically coupled to operate in a bipolar modality to deliver RF energy to the treatment cavity in a specific direction and at a specific angle so that the bipolar RF electrodes of the second set selectively target RF energy to at least one of: a specific layer, a specific depth, and/or a specific location or depth within a specific layer of a second zone within the target tissue. The second set of bipolar RF electrodes targets RF energy configured in accordance with one or more parameters to treat the second zone of the target tissue. The first set of bipolar RF electrodes produces a treatment effect in the first zone different from the treatment effect the second set of bipolar RF electrodes produces in the second zone. The RF energy the first set of bipolar RF electrodes targets to the first zone may be different from RF energy the second set of bipolar RF electrodes targets to the second zone. In one configuration, the treatment effects in the first zone and in the second zone of the target tissue are fractional treatment effects.

In one configuration, the at least two bipolar RF electrodes of the first set are disposed in a transverse orientation relative to one another on opposite surfaces of the treatment cavity. In a further configuration, the at least two bipolar RF electrodes of the second set are disposed in a transverse orientation relative to one another on opposite surfaces of the treatment cavity. The first set, and/or the second set, of at least two bipolar RF electrodes may include at least two fractionated bipolar RF electrodes including one or more RF fractions.

One or more of the multiple bipolar RF electrodes are configured so that electronic coupling of the bipolar RF electrodes can switch, wherein electronic coupling of one of the bipolar RF electrodes of the first set can switch to electronically couple with one of the bipolar RF electrodes of the second set to change the specific direction and the specific angle of RF energy conveyed to the treatment cavity.

Each bipolar RF electrode of the multiple bipolar RF electrodes is disposed at an angle relative to the treatment cavity to facilitate contact between the bipolar RF electrode and the target tissue.

Implementations of the invention may also include one or more of the following features. The first set of bipolar RF electrodes delivers RF energy to the treatment cavity in the specific direction and at the specific angle to target RF energy along an X axis and along a Z axis of at least one of: the specific layer, the specific depth, and/or the specific location or depth of the first zone within the target tissue. The first set of bipolar RF electrodes may also deliver RF energy to the treatment cavity in the specific direction and at the specific angle to target RF energy along a Y axis of at least one of: the specific layer, the specific depth, and/or the specific location or depth of the first zone within the target tissue. RF energy the first set of RF electrodes targets to the first zone produces an RF induced heating profile within the first zone.

Similarly, the second set of bipolar RF electrodes delivers RF energy to the treatment cavity in the specific direction and at the specific angle to target RF energy along an X axis and along a Z axis of at least one of: the specific layer, the specific depth, and/or the specific location or depth of the second zone within the target tissue. The second set of bipolar RF electrodes may also deliver RF energy to the treatment cavity in the specific direction and at the specific angle to target RF energy along a Y axis of at least one of: the specific layer, the specific depth, and/or the specific location or depth of the second zone within the target tissue. The second set of RF electrodes targets to the second zone produces an RF induced heating profile within the second zone.

The RF induced heating profile within the first zone produces one or more treatment effects different from the one or more treatment effects the RF induced heating profile within the second zone produces. The first and the second set of bipolar RF electrodes thereby selectively produce tissue specific and tissue depth specific treatment effects.

Implementations of the invention may further include one or more of the following features. The system includes at least two ultrasound transducers disposed within the treatment applicator and along predetermined internal surfaces that define the treatment cavity. Each ultrasound transducer is disposed at an angle relative to the treatment cavity and is configured to deliver ultrasound energy in a specific direction and at a specific angle to target ultrasound energy to at least one of: a specific layer, a specific depth, and/or a specific location or depth within a specific layer a volume of the target tissue. The ultrasound transducers may include at least two fractionated ultrasound transducers including one or more ultrasound fractions or sub-components. The system further includes an ultrasound energy source operatively coupled to the ultrasound transducers.

The ultrasound transducers produce one or more treatment effects within at least one of: the specific layer, the specific depth, and/or the specific location or depth within the specific layer of a volume of the target tissue. Such one or more treatment effects the ultrasound transducers produce may be different from the treatment effects the first set of bipolar electrodes produces in the first zone and the second set of bipolar electrodes produces in the second zone.

The system also includes a PC and a microprocessor configured to operate the first and the second sets of paired bipolar RF electrodes and the ultrasound transducers. The PC and the microprocessor operate the first and the second sets of paired bipolar RF electrodes and the ultrasound transducers in at least one of: a continuous mode and a pulsed mode.

In another aspect, the invention provides a system for fractional treatment of a condition or pathology of a tissue including a treatment applicator disposed and configured to deliver treatment energy to a target tissue. The treatment applicator defines within its interior a treatment cavity to engage the target tissue. The system includes multiple ultrasound emitting devices disposed within the treatment applicator and along predetermined internal surfaces that define the treatment cavity. The system also includes an ultrasound energy source operatively coupled to the multiple ultrasound emitting devices. Each ultrasound emitting device is disposed at an angle relative to the treatment cavity and is configured to generate ultrasound energy. Multiple ultrasound emitting devices include a first set of at least two ultrasound emitting devices disposed along at least two internal surfaces of the treatment cavity. The at least two ultrasound emitting devices are disposed to deliver ultrasound energy to the treatment cavity in a specific direction and at a specific angle so that the at least two ultrasound emitting devices of the first set selectively target ultrasound energy to at least one of: a specific layer, a specific depth, and/or a specific location or depth within a specific layer of a first zone within a volume of the target tissue.

The system can further include a second set of at least two ultrasound emitting devices disposed along at least two internal surfaces of the treatment cavity. The at least two ultrasound emitting devices are disposed to deliver ultrasound energy to the treatment cavity in a specific direction and at a specific angle so that the at least two ultrasound emitting devices of the second set selectively target ultrasound energy to at least one of: a specific layer, a specific depth, and/or a specific location or depth within a specific layer of a second zone of the volume of the target tissue.

The treatment effects the first set of ultrasound emitting devices produces in the first zone of the target tissue can be different from the treatment effects the second set of ultrasound emitting devices produces in the second zone of the target tissue. The first and the second set of ultrasound emitting devices thereby selectively produce tissue specific and tissue depth specific treatment effects.

In a further aspect, the invention provides a treatment applicator for providing fractional treatment of a condition or pathology of a tissue. The treatment applicator defines a treatment cavity within its interior that is configured to engage a three-dimensional volume of target tissue. Multiple bipolar RF electrodes are disposed along internal surfaces defining the treatment cavity. Each bipolar RF electrode is disposed at an angle relative to the treatment cavity. An RF energy source operatively couples to the multiple bipolar RF electrodes. At least two RF electrodes are disposed along at least two internal surfaces of the treatment cavity. The at least two RF electrodes are electronically coupled to operate in a bipolar modality to deliver RF energy to the treatment cavity in a specific direction and at a specific angle so that the bipolar RF electrodes selectively target RF energy to at least one of: a specific layer, a specific depth, and/or a specific location or depth within a specific layer to produce one or more different RF treatment effects within the volume of target tissue.

The treatment applicator may further include at least two ultrasound transducers disposed along at least two internal surfaces of the treatment cavity. The at least two ultrasound transducers are configured to deliver ultrasound energy to the treatment cavity in a specific direction and at a specific angle to target ultrasound energy to at least one of: a specific layer, a specific depth, and/or a specific location or depth within a specific layer to produce one or more different ultrasound treatment effects within the volume of target tissue.

### BRIEF DESCRIPTION OF THE DRAWINGS

The drawings are for purposes of illustrating aspects of the invention and are not rendered to any particular or accurate scale.

FIG. 1 is a schematic diagram of one aspect of the invention including a system for skin and tissue treatment employing radiofrequency (RF) treatment energy;

FIG. 2 is a schematic diagram of another aspect of the invention including a system for skin and tissue treatment employing at least RF treatment energy and, optionally, in combination with ultrasound treatment energy;

FIG. 3 is a cross-sectional diagram of a prior art RF treatment applicator;

FIG. 4 is a cross-sectional diagram of another aspect of the invention including an RF treatment applicator;

FIG. 5A is a cross-sectional diagram of the treatment applicator shown in FIG. 4 with relative tissue resistivity of target tissue illustrated;

FIG. 5B is a graph illustrating a potential thermal (heating) profile achieved with prior art straight parallel RF electrodes, and a potential thermal (heating) profile achieved with sloped or tilted RF electrodes included in the treatment applicator shown in FIG. 4;

FIG. 6 is a perspective diagram of one configuration of bipolar RF electrodes included in the treatment applicator shown in FIG. 4;

FIG. 7A is a cross-sectional diagram of the treatment applicator shown in FIG. 4 illustrating potential patterns at which the applicator delivers RF energy to target tissue;

FIG. 7B is a perspective diagram of one configuration of bipolar RF electrodes included in the treatment applicator shown in FIG. 7A;

FIG. 8 is a cross-sectional diagram of the treatment applicator shown in FIG. 4 illustrating other potential patterns at which the applicator delivers RF energy to target tissue;

FIG. 9 is a chart illustrating a calculated model of a distribution and depth of RF induced heating of a given thermal (heating) profile per tissue layer and per tissue depth;

FIG. 10 is a cross-sectional diagram of another aspect of the invention including a treatment applicator configured with RF electrodes and with ultrasound emitting devices to provide a multi-modality treatment;

FIG. 11 is a perspective diagram of one configuration of bipolar RF electrodes included in the treatment applicator shown in FIG. 10; and

FIG. 12 is a perspective diagram of one configuration of ultrasound emitting devices included in the treatment applicator shown in FIG. 10.

### DETAILED DESCRIPTION

A tissue treatment system, device and methods provide multi-modality treatment of skin, tissue and organ conditions and pathologies using different types of treatment energy, alone or in combination, including radiofrequency (RF) energy and ultrasound energyto achieve desired tissue-specific effects. The system, device, and methods according to the invention selectively and fractionally treat three-dimensional volumes of skin or other soft tissue with one or more energy types and are configured to precisely target and deliver measured treatment energy to specific tissue zones, e.g., specific tissue types, layers and/or depths, within a given volume of skin and tissue. The one or more types of treatment energy can thereby more accurately affect the structure or activity of different and specific tissue types, layers and/or depths within a given tissue zone in accordance with the desired or required impact such treatment will have in a particular tissue layer and/or a particular tissue depth, while other layers or depth of target can be treated differently. The system, device, and methods employ a treatment applicator constructed and arranged to engage a three-dimensional volume of skin or tissue and to deliver one or more types of treatment energy to the engaged volume of skin or tissue, such that, different zones, layers and/or depths of the skin or tissue are treated selectively with one or more types of energy to achieve tissue or zone-specific treatment effects.

The treatment applicator is equipped with various combinations and arrangements of RF electrodes and/or ultrasound transducers to deliver controllable energy types to specific tissue zones. The energy-emitting elements thereby deliver a selected energy type at a certain energy intensity or fluence, or range of energy intensities or fluence, for certain duration to a specific tissue type, layer and/or depth within a given volume of target tissue. Multiple energy-emitting elements are positioned and arranged along an internal treatment chamber defined within the interior of the treatment applicator to deliver treatment energy to the target tissue in a three-dimensional pattern.

For instance, the treatment applicator and energy-emitting elements, such as one or more paired RF electrodes, may be arranged to operate and deliver RF energy to a specific, e.g., predetermined tissue layer and to a specific location or depth within the tissue layer of a given three-dimensional tissue volume. Where the volume of target tissue is positioned relative to, e.g., between, the RF electrodes, the particular arrangement and operation of the RF electrodes can supply RF energy to the specific tissue layer to produce a treatment impact along an X-axis, Z-axis and/or Y-axis of the tissue layer. The RF electrodes can be further arranged and operated to supply RF energy along specific depths within the tissue layer to produce a treatment impact along a Y-axis of the tissue layer. In addition, the RF electrodes may be configured and arranged to provide fractional RF treatments to a specific tissue layer and to specific locations and depths within the tissue layer to provide selective fractional treatments to different zones within the specific tissue layer. In this case, the RF electrodes can be configured and arranged within the treatment chamber to selectively deliver RF energy to specific zones within the tissue layer, such that, a specific fractional treatment impact is produced in a particular zone of the tissue layer that may be different from the fractional treatment impacts produced in other zones of the tissue layer. For instance, a certain deep zone within the tissue layer may be treated selectively with RF energy to produce relatively high temperatures within the deep zone to achieve ablation or cellular destruction. A comparatively mid-depth zone within the same tissue layer may be treated selectively with RF energy to produce a relatively lower temperature of the tissue layer to achieve blood vessel coagulation, and another zone of relatively superficial depth of the same tissue layer may be treated selectively with RF energy to produce within the superficial zone a relatively high temperature to achieve cell (fibroblast) stimulation.

As an example, in skin rejuvenation treatments the treatment applicator and a particular arrangement of RF electrodes according to the invention selectively target and deliver RF energy to different layers of the dermis and hypodermis to produce a specific desired thermal response and treatment impact within each layer of the dermis and hypodermis. The arrangement and operation of RF electrodes allows the treatment applicator to selectively deliver RF energy at different intensities and fluences, or different ranges of energy intensities and fluences, depending on the type of tissue layer, and/or location or depth within the layer that is targeted for treatment. The RF electrodes may be arranged to target RF energy configured with a given energy intensity or fluence, or intensities or fluences within a given range, to the reticular dermal layers (deep tissue zone) of a skin sample to produce the desired thermal response and impact of denaturing and thereby shrinking collagen. The RF electrodes may also be arranged to target RF energy configured with a different energy intensity or fluence, or intensities or fluences within a different range, to the papillary dermal layers (less deep tissue zone) of the skin sample to produce the desired (and different) thermal response and impact of stimulating fibroblasts for new collagen formation. Further, the RF electrodes may be arranged to target RF energy configured with a different intensity or fluence, or intensities or fluences within a different range, than that delivered to the papillary and dermal layers to the hypodermal layer or subcutaneous fat layers (relatively deepest tissue zone) of the skin sample to produce the desired (and different) thermal response and impact of cellular and/or extra cellular matrix (ECM) destruction for treatment of cellulite. The RF electrodes are configured to deliver RF energy to each layer of the dermis and hypodermis concomitantly and/or sequentially.

In addition, the RF energy may be delivered fractionally in this example to the dermal layers, such that, areas of the dermal layers are untreated and remain intact to facilitate a blood supply through the skin sample and to the skin surface. Fractional treatment using the particular configurations and arrangements of RF electrodes within the treatment applicator according to the invention thereby enables a number of different treatment (energy) impacts within different zones of a given sample of target tissue that are specific to a particular tissue type/layer and/or are specific to particular depths within a tissue type/layer.

The treatment applicator is configured to engage with a three-dimensional volume of target skin or tissue by various mechanical and/or pressure techniques. As described above, the combinations of light-emitting components, RF electrodes and/or ultrasound transducers of the treatment applicator, and their specific arrangements and positions within the treatment applicator, deliver treatment energy to one or more specific tissue zones within the engaged volume of target skin or tissue. The arrangements and positions of the components, electrodes and/or transducers relative to one another within the treatment applicator and relative to the tissue zone(s) of a target tissue help to enable creation of reasonably predictable thermal (heating) profiles within the tissue zone(s), such that, one or more types of treatment energy may be precisely targeted and specifically delivered to the tissue zone(s) to affect certain tissue types and layers at certain depths. Thermal profiles may be manipulated using the system, device and methods according to the invention in order to enable profiles to relate closely to a particular tissue zone or to a particular tissue type, layer, and depth. More specifically, thermal profiles may be manipulated through use of different types, combinations, arrangements, and positions of the light-emitting components, RF electrodes, and ultrasound transducers within the treatment applicator as well as through different modes of operation of such components, electrodes, and transducers. The system, device, and methods according to the invention may be constructed and arranged to deliver fractional treatments, as well as homogeneous treatments, to tissue zones within an engaged volume of skin or other soft tissue.

Similarly, the treatment applicator according to the invention may configure and apply ultrasound energy, alone or in combination with RF energy, to a three-dimensional volume of target tissue to achieve multiple and different responses along an X axis, a Z axis, and/or a Y axis of the target tissue to produce specific and different treatment impacts that are specific to the type, layer and/or depth of the targeted tissue. In this case, the treatment applicator according to the invention provides multi-modality treatment. Further, the treatment applicator according to the invention may configure and apply RF energy alone or in combination with ultrasound energy to a three-dimensional volume of target tissue to produce specific and different treatment impacts.

The treatment system, device, and methods according to the invention can be constructed and configured to provide external treatment to superficial tissue. Alternatively, the system, device and methods according to the invention can be constructed and configured to provide treatment through minimally invasive procedures and techniques for treatment of deep, internal tissue, wherein a treatment applicator would be constructed and designed as a catheter-like device, endoscope or laparoscope. Other embodiments are within the scope of the invention.

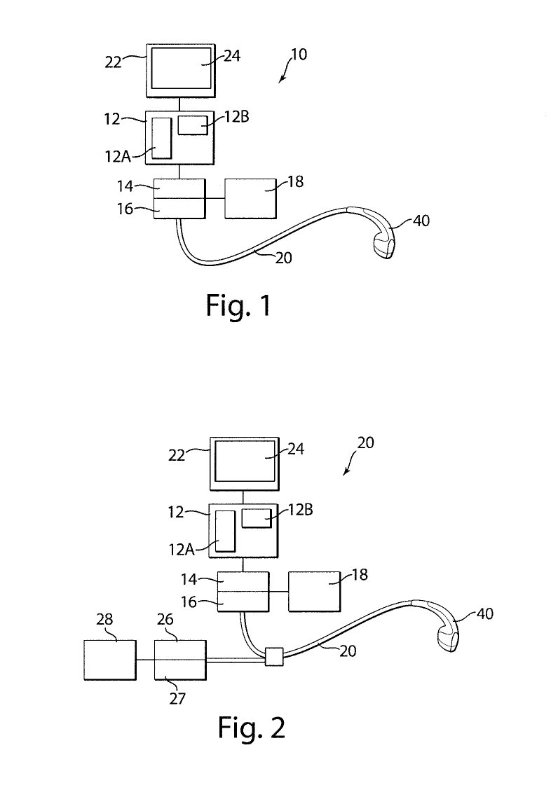

Referring to FIG. 1, in an aspect, the invention provides a system 10 for delivering RF treatment energy to one or more tissue zones within a given three-dimensional volume of skin or soft tissue to target RF energy to one or more specific skin or tissue layers and/or to one or more specific locations or depths within the skin or tissue layers. Targeting RF energy to specific layers and/or specific locations or depths within layers enables the system 10 according to the invention to treat different skin or tissue layers and different locations or depths within layers selectively and differently to produce different treatment effects that are specific to a particular layer and/or a particular location or depth within the layer. The system 10 thereby provides multiple and different RF treatments within a given three-dimensional volume of target tissue that are precisely targeted and controlled in accordance with the type and location of treatment sites within the volume of target tissue and the treatment impact desired or required at each treatment site.

The system 10 includes a control unit 12 comprising a PC 12A and a microprocessor 12B operatively coupled to control treatment in accordance with treatment parameters that may be pre-set and/or pre-programmed, and/or that may be configured and set with the control unit 12 by an operator of the system 10, to deliver different types of treatment and different treatment protocols. The system 10 further includes an RF source including a signal generator 14 operatively coupled to a signal amplifier 16 and a power supply 18, and being configured to produce signals and to deliver electrical current at given frequencies and power to RF energy-emitting devices disposed within a treatment applicator 40. The treatment applicator 40 is operatively connected to these system 10 components, e.g., via an umbilical cable 20, and is configured to administer RF energy produced by the RF energy-emitting devices to target tissue. As described below with reference to FIG. 4, the treatment applicator 40 is equipped with RF energy-emitting devices configured and arranged within the treatment applicator 40 to target RF energy to selected skin and soft tissue layers and/or selected locations or depths within such layers to produce different types of treatment. The treatment applicator 40 is operatively connected to a vacuum pump (not shown) and is further configured to deliver a negative pressure vacuum to a treatment area of skin or tissue to deform and draw a three-dimensional volume of target tissue into a treatment cavity defined within the interior of the treatment applicator 40, as described below with reference to FIG. 4.

The system 10 may further include an LCD monitor 22 and a touch screen 24 operatively coupled and configured to serve as a user interface for receiving inputs to activate and operate the system 10. In addition, the system 10 may further include one or more contact monitoring devices to verify contact between energy-emitting devices of the treatment applicator 40 and target tissue, one or more vacuum monitoring devices to verify a given pressure vacuum level is reached and maintained, and one or more power monitoring devices to verify power of treatment energy the system 10 delivers. The system 10 components, with the exception of the treatment applicator 40 and the umbilical cable 20, may be disposed within a unitary housing or console constructed and arranged for portability and/or for a permanent installation.

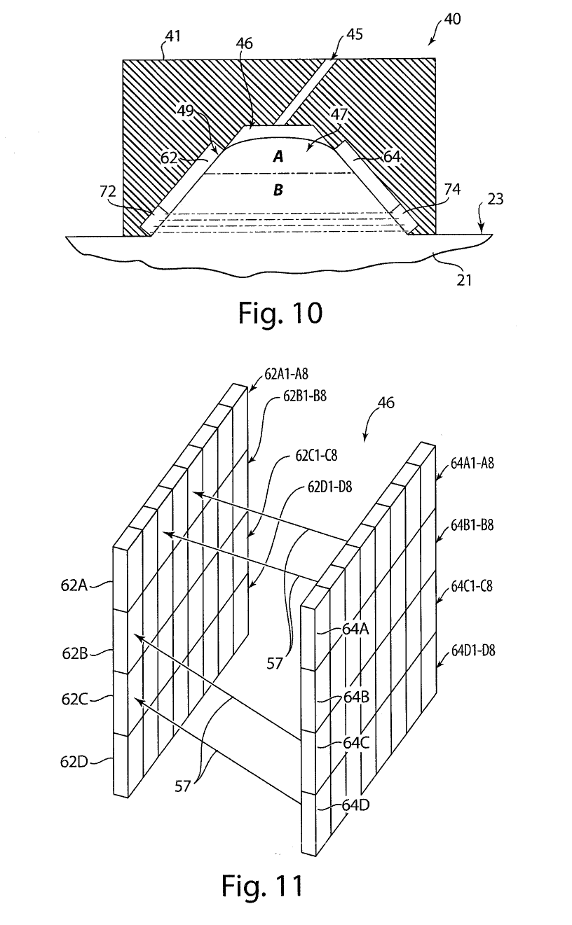

Referring to FIG. 2, in another aspect, the invention provides a system 20 for multi-modality treatment constructed and arranged for providing RF energy and treatment, as described above with reference to FIG. 1, and further constructed and arranged for providing ultrasound energy and treatment to one or more tissue zones within a given three-dimensional volume of skin or soft tissue. The system 20 is configured to target RF energy and is further configured to target ultrasound energy to one or more specific skin or soft tissue layers and/or to one or more specific locations or depths within the skin or tissue layers. In addition to the components shown in and described with reference to FIG. 1, the system 20 further includes an ultrasound energy source including a signal generator 26 operatively coupled to a signal amplifier 27 and a power supply 28, and being configured to produce signals and to deliver electrical current at given frequencies and power to ultrasound emitting devices, e.g., ultrasound transducers, disposed within the treatment applicator 40 to administer ultrasound energy to target tissue. As described below with reference to FIG. 10, the treatment applicator 40 is equipped with RF energy-emitting devices and with ultrasound emitting devices, which are configured and arranged for targeting ultrasound energy to a selected skin or soft tissue layer and/or a selected location or depth within the layer. This configuration of the treatment applicator 40 according to the invention may thereby provide different types of treatment and may produce different desired or required treatment impacts that are specific to a particular skin or soft tissue layer and/or a particular location or depth within the layer. Within a given volume of target tissue, the treatment applicator 40 can deliver RF treatment to one type of skin or soft tissue layer, and/or a particular location or depth within that layer, and can also deliver ultrasound treatment to a different type of skin or soft tissue layer, and/or a particular location or depth within this layer, to achieve multiple and different treatment effects.

The systems 10 and 20 according to the invention shown in FIG. 1 and FIG. 2 can operate in a manual and/or an automatic mode to deliver RF and/or ultrasound treatment to target tissue. Additionally, or alternatively, the systems 10 and 20 may operate in a scanning mode, wherein RF energy and/or ultrasound energy are delivered to target tissue in accordance with a predetermined plan and/or pattern. Further, the systems 10 and 20 may operate in any of the noted modes in accordance with feedback provided by one or more monitoring elements operatively coupled with the systems 10 and 20 to provide feedback input and/or data related, but not limited to, a location of a treatment site or zone, one or more actual treatment effects occurring/occurred in a particular treatment site or zone, temperature of a treatment site or zone and the impedance or conductivity at a treatment site or zone.

The systems 10 and 20, and the treatment applicator 40, according to the invention may be constructed and arranged to provide multi-modality treatment for aesthetic, therapeutic, or surgical purposes to skin, soft tissue or organs.

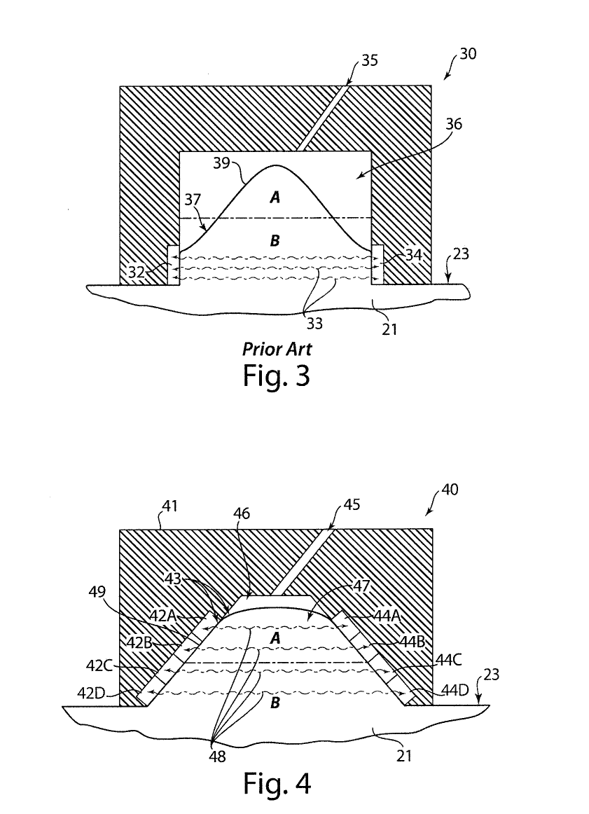

Referring to FIG. 3 and FIG. 4, in a further aspect, the invention provides the treatment applicator 40 configured for use with any of the systems 10 and 20 shown in and described with reference to FIGS. 1 and 2. The treatment applicator 40 according to the invention shown in FIG. 4 includes at least two energy-emitting elements including RF electrodes and/or ultrasound emitting devices, e.g., transducers to provide multi-modality treatment for tissue-specific and depth-specific treatment effects.

FIG. 3 is a cross-sectional diagram of a prior art treatment applicator 30 including two straight bipolar RF electrodes 32 and 34 disposed along each side of a treatment cavity 36 defined within the interior of the treatment applicator 30. The bipolar RF electrodes 32 and 34 are positioned in a substantially parallel orientation relative to one another. The area of each RF electrode 32 and 34 that emits/receives RF energy is positioned substantially straight and at a substantially parallel orientation relative to the treatment cavity 36 and relative to a volume of tissue, e.g., Zone A or Zone B, engaged within the cavity 36 for treatment. The RF electrodes 32 and 34 operate in a closed circuit configuration whereby RF energy propagates and flows between the two electrodes, and also through a volume of tissue, Zone A or Zone B, as indicated by lines 33 shown in FIG. 3, when the tissue is disposed at a certain depth within the cavity 36. The flow of RF energy is limited to or defined by the area between the RF electrodes 32 and 34.

The treatment applicator 30 is configured to generate and apply a negative pressure vacuum 35 to the treatment cavity 36 in order to drawn into the cavity 36 a given volume of tissue 37 when the treatment applicator 30 is placed on a surface 23 of the skin 21. Introduction of the volume of tissue 37 into the cavity 36, using pressure vacuum techniques (or a mechanical device and/or techniques) results in the volume of tissue 37 engaged within the cavity 36 forming a Gaussian-like or sloped shape. The Gaussian-like or sloped shape of the tissue volume 37 inhibits or prevents sufficient contact between a surface 39 of the tissue volume 37 and the contact areas of the RF electrodes 32 and 34, such that, the surface 39 of the tissue volume 37 is only partially in contact with the RF electrodes 32 and 34. As illustrated in FIG. 3, Zone B of the tissue volume 37 is positioned substantially in contact with the RF electrodes 32 and 34, while Zone A of the tissue volume 37 has little contact with the RF electrodes 32 and 34 due to the Gaussian-like shape of the tissue volume. Actual contact between the surface 39 of the tissue volume 37 and the RF electrodes 32 and 34, and thereby coverage of the RF electrodes 32 and 34, facilitates delivery of RF energy and enables flow of RF energy through the tissue volume 37. This occurs as a result of the inherent impedance or resistivity of the tissue volume 37 in contact with the RF electrodes 32 and 34. However, with insufficient contact between the surface 39 of the tissue volume 37 and the RF electrodes 32 and 34, as shown in FIG. 3, the RF electrodes are only partially covered. As a result, the electrodes deliver RF energy into the cavity 36 in an uncontrolled manner. In addition, due to low impedance to the RF energy, high energy density and excessive fluence are created within the cavity 36. Such high energy density and excessive fluence may have an undesirable intense affect on superficial tissue, such as tissue within Zone A, of the engaged volume of tissue 37.

In addition, the configuration of the prior art treatment applicator 30 including the straight RF electrodes 32 and 34 cannot deliver RF energy in a controlled manner to specific depths or locations of the volume of tissue 37 engaged within the cavity 36. Rather, the RF energy delivered to any depth within the volume of target tissue 37 is limited to the extent that a specific depth of the volume of tissue 37 is drawn sufficiently into the cavity 36, such that, the specific location or depth is positioned adequately between the RF electrodes 32 and 34 to enable flow of RF energy through the tissue location or depth. Further, the RF electrodes 32 and 34 are not configured to selectively deliver fractional RF energy to specific locations or depths of the volume of tissue 37 and thereby cannot achieve different fractional treatment responses that are layer-specific and/or depth-specific.

In contrast, FIG. 4 illustrates a cross-sectional diagram of the treatment applicator 40 according to the invention including a treatment cavity 46 defined within the interior of the applicator 40 and including at least two tilted bipolar RF electrodes, and preferably multiple tilted bipolar RF electrodes 42A-42D and 44A-44D. Each RF electrode is designed and configured to electronically couple with one or more other RF electrodes to function in a bipolar modality conducting RF current between electrodes and thereby through the treatment cavity 46.

Electronically coupled bipolar RF electrodes are located along internal surfaces of the treatment applicator 40 that define the treatment cavity 46 and may be positioned relative to one another in any of a variety of arrangements to deliver RF energy in a number of different directions and at a number of different angles to target RF energy to specific tissues (layers) and specific tissue depths along the X, Z, and/or Y axes of a given three-dimensional volume of target tissue 47. For instance, one bipolar RF electrode, such as RF electrode 42A, may be located along one side of the treatment cavity and electronically couple or “pair” with one or more other RF electrodes, such as RF electrodes 44A, 44B and/or 44C, to convey RF energy in a number of different directions and at a number of different angles to target specific tissues (layers) and specific tissue depths along the X, Z, and/or Y axes of the tissue volume 47. As used to disclose the invention, “paired” bipolar RF electrodes refers to two or more electronically coupled bipolar RF electrodes, RF electrode “pairings” refers to two or more electronically coupled bipolar RF electrodes, and “pairing” RF electrodes refers to electronically coupling two or more bipolar RF electrodes. In addition, the bipolar RF electrodes are configured and operated to permit switching of electronic coupling of RF electrodes, such that, electronic couplings between a given bipolar RF electrode and one or more other RF electrodes may be switched to electronically couple the given bipolar RF electrode with one or more different RF electrodes. Switching electronic couplings of the bipolar RF electrodes 42A-42D and 44A-44D and their operation permit the treatment applicator 40 according to the invention to configure and to target RF energy in multiple directions and multiple angles.

FIG. 4 shows an illustrative arrangement of electronically coupled or paired bipolar RF electrodes 42A-42D and 44A-44D positioned along internal surfaces of the treatment applicator 40. In this configuration, one set of RF electrodes 42A-42D is disposed along one side of the treatment cavity 46 opposite to another set of RF electrodes 44A-44D disposed along an opposing side of the cavity 46 to position RF electrode sets 42A-42D and 44A-44D at a substantially transverse orientation to one another across the cavity 46. Alternatively, one or more of the RF electrodes 42A, 44A, 42B, 44B, etc. may be disposed at an offset position relative to one or more other RF electrodes (not shown) along different sides of the cavity 46. As will be described below, one or more RF electrodes 42A-44D of one set may be electronically coupled or paired with one or more RF electrodes 44A-44D of the other set for operation in a bipolar modality.

The invention is not limited to the configuration of the electronically coupled RF electrodes as described with reference to FIG. 4 and anticipates that multiple bipolar RF electrodes 42A-42D and 44A-44D may be positioned in any of a variety of arrangements along any of the internal surfaces that define the treatment cavity 46 and such RF electrodes may be electronically coupled in any of a variety of coupling configurations.

The applicator 40 includes a housing 41 and is further configured to permit application of a negative pressure 45 to the treatment cavity 46, and/or to permit use of a mechanical device and/or technique, capable of engaging a three-dimensional volume of target tissue 47 within the cavity 46. The applicator 40 applies a given negative pressure Pₓ to the treatment cavity 46 to create a pressure vacuum sufficient to draw into the cavity 46 the volume of target tissue 47 to a desired or required depth within the cavity 46, such that, the pressure vacuum positions one or more specific tissues (layers) and/or tissue depths of the target tissue 47 between the multiple tilted RF electrodes 42A-42D and 44A-44D to receive treatment.

Each RF electrode 42A-42D and 44A-44D is disposed at a sloped or tilted position and at a specific angle relative to the cavity 46 and relative to the volume of target tissue 47 that the cavity 46 engages for treatment. In addition, the area of each RF electrode 42A-42D and 44A-44D configured to emit/receive RF energy is positioned at a specific angle relative to the cavity 46 and the volume of target tissue 47. The size and shape of emitting/receiving areas of each RF electrode may be designed and configured to correspond to and to accommodate the structure and anatomy, e.g., size and/or shape, of the volume of target tissue 47 being treated in order to facilitate and ensure contact between the target tissue 47 and emitting/receiving areas of the RF electrodes 42A-42D and 44A-44D.

In addition, specific angles of the multiple tilted bipolar RF electrodes 42A-44D and 44A-44D relative to the cavity 46 and the target tissue 47, such as, for instance, relative to a surface 49 of the tissue volume 47, help to ensure contact and facilitate the precision of contact between each RF electrode and the target tissue 47, e.g., tissue surface 49, such that, emitting/receiving areas of the RF electrodes 42A-44D and 44A-44D are covered or are substantially covered, e.g., minimal emitting/receiving area of RF electrode 42A-44D and 44A-44D is exposed. The number and angles of the tilted RF electrodes 42A-44D and 44A-44D thereby help to accommodate the Gaussian-like or sloped shape of the volume of target tissue 47 when the treatment applicator 40 engages the tissue 47 within the cavity 46. For instance, in contrast to the prior art applicator 30 shown in FIG. 3, more contact between the tilted RF electrodes 42A-44D and 44A-44D and the surface 49 of the target tissue 47 in proximity to Zone A may be accomplished with the treatment applicator 40 according to the invention. As a result, the treatment applicator 40 and multiple tilted RF electrodes 42A-44D and 44A-44D provide controlled delivery of RF energy to the surface 49 of the target tissue 47 with reliable and predictable energy density and fluence. In addition, the treatment applicator 40 eliminates or substantially minimizes the occurrence of high energy densities and excessive fluence within the treatment cavity 46.

A coupling medium or material 43 may be optionally disposed along the surface 49 of the volume of target tissue 47, depending on the energy level to be delivered to the tissue volume, to assist in achieving contact between the RF electrodes 42A-42D and 44A-44D and the volume of target tissue 47, and/or to aid in establishing conductivity between the RF electrodes 42A-42D and 44A-44D and the target tissue 47. In particular, coupling material 43 assists in conducting RF energy, and ultrasound energy as described below, from the RF electrodes 42A-42D and 44A-44D to the surface 49 of and through the volume of target tissue 47. Such coupling material 43 may include a lotion or gel applied directly to the surface 49 of the target tissue volume 47, such that, the lotion or gel is disposed between the RF electrodes 42A-42D and 44A-44D and the volume of target tissue 47.

Operating in a bipolar modality the tilted RF electrodes 42A-42D and 44A-44D are designed and configured to propagate RF current through the treatment cavity 46 between electronically coupled or paired bipolar RF electrodes 42A-44D and 44A-44D RF, as illustrated by lines 48 shown in FIG. 4. As RF current flows between electrodes, RF energy applies to the surface 49 of the target tissue 47.

Parameters controlling operation of the treatment applicator 40 and the tilted RF electrodes 42A-42D and 44A-44D, as well as the specific angles and directions with which the RF electrodes convey RF energy, help target RF energy to particular tissues (layers) and/or particular locations or depths within the target tissue volume 47. Each RF electrode 42A-44D and 44A-44D is configured and operated by the treatment applicator 40 to deliver RF current to the target tissue 47 in a specific direction and at a specific angle, such that, RF energy flows through the tissue 47 in a specific direction and at a specific angle to thereby selectively induce heating in a particular tissue (layer) and/or tissue depth of the tissue volume 47. As a result, certain thermal treatment responses and effects are produced that are specific to the particular tissue (layer), the particular depth, and/or a particular location or depth within a given tissue (layer). The positions and angles of the tilted RF electrodes 42A-44D and 44A-44D, and their pairings and operation, thereby enable the treatment applicator 40 to target RF energy to different tissues (layers) and/or different tissue depths to produce selective and different RF treatments. In addition, the RF energy that one or more RF electrodes 42A-42D and 44A-44D deliver can be configured with the same or different characteristics, e.g., energy fluence, to selectively and differently treat particular tissues (layers) and/or tissue depths. The treatment applicator 40 according to the invention can thereby deliver RF energy with the same or different characteristics, and can target RF energy to produce multiple and different treatments in different tissues (layers), depths, and/or locations or depths of a given tissue layer within a given three-dimensional volume of target tissue 47.

For instance, as the configuration and arrangement of the multiple bipolar RF electrodes of FIG. 4 illustrates one or more RF electrodes, e.g., 42A and 44A electrodes and 42B and 44B electrodes, may be electronically coupled or paired and may operate to deliver RF energy in a specific direction and at a specific angle to target a particular tissue (layer) and/or location or depth within Zone A. RF energy delivered to Zone A by RF electrodes 42A, 44A and 42B, 44B would be configured with one or more characteristics, e.g., energy fluence, to selectively treat the targeted tissue (layer) and/or targeted location or depth within Zone A and to produce tissue (layer) specific and/or depth specific treatment responses. Similarly, a particular tissue (layer) and/or depth within Zone B may be targeted and selectively treated with RF energy provided by one or more electronically coupled or paired RF electrodes, e.g., 42C and 44C electrodes and 42D and 44D electrodes, that operate to deliver RF energy in a specific direction and at a specific angle to Zone B to produce specific treatment responses. Treatment responses in Zone B may be different from treatment responses in Zone A. Similarly, RF energy delivered by to Zone B by RF electrodes 42C, 44C and 42D, 44D would be configured with one or more characteristics, e.g., energy fluence, that are the same or different from the RF energy delivered to Zone A to selectively treat the target tissue (layer) and/or depth.

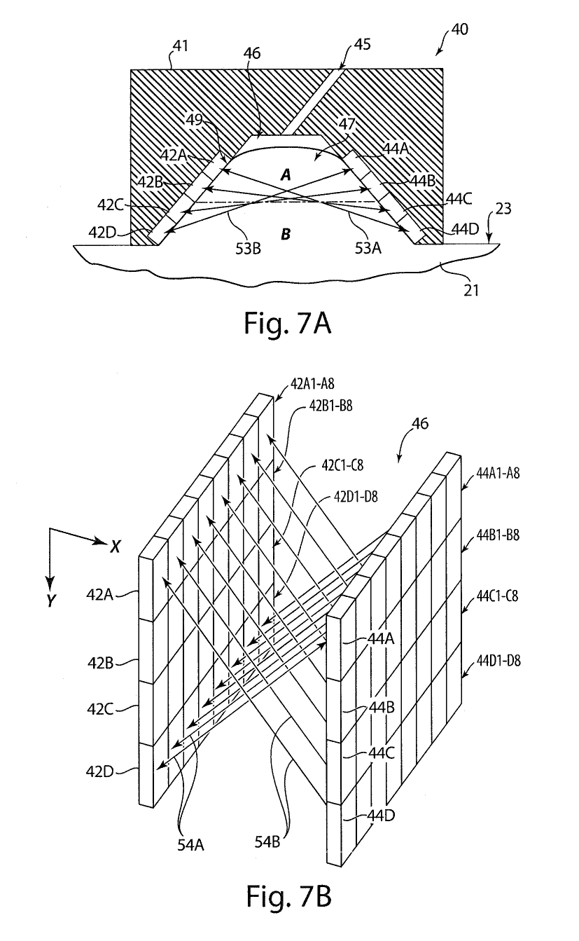

As a result, the multiple tilted RF electrodes 42A-42D and 44A-44D can thereby precisely control the depth and distribution of RF induced heating within the target tissue 47 along the X, Z and/or Y axes. In addition, by delivering RF energy in specific directions and at specific angles, the multiple tilted RF electrodes 42A-44D and 44A-44D can also help to achieve RF induced treatment in deep layers of a given volume of target tissue 47. As described in detail below with reference to FIGS. 7A and 7B, the multiple tilted RF electrodes 42A-44D and 44A-44D help to produce heating profiles that conform to reasonably predictable heating profiles that are based, at least in part, on models of uniform RF energy resistivity of various tissues and tissue depths of a given volume of target tissue 47. The treatment head 40 according to the invention can thereby provide multiple and different three-dimensional treatments to a given volume of target tissue 47, producing controlled and customized thermal treatment responses that are specific to the tissue (layer) and/or the tissue depth targeted.

The parameters that the system 10 and 20, and/or the treatment applicator 40, may use to configure RF energy the one or more RF electrodes 42A-42D and 44A-44D target to specific tissues (layers) and specific depths may include, but are not limited to, the current level of the RF electrodes 42A-42D and 44A-44D, the power and the peak power, the frequency of the RF energy, the intensity or fluence of the RF energy applied to specific tissues (layers), specific tissue depths and/or specific locations or depths within a given tissues (layers), and the duration or length of time of exposure of target tissue to RF energy. In addition, as mentioned, the inherent impedance or resistivity of a particular target tissue type, layer and/or depth may be considered and employed by the system 10 and 20, and/or the treatment applicator 40, in configuring RF energy.

The specific angles of slope or tilt of the RF electrodes 42A-42D and 44A-44D relative to the treatment cavity 46 may be altered in accordance with, for instance, a patient's skin or tissue characteristics, the specific area to be treated, the tissue (layer) and/or location or depth within the layer targeted for treatment, and/or the tissue depth targeted for treatment within a given volume of target tissue 47, as well as in accordance with one or more parameters to produce the treatment impact desired or required within the target tissue 47. In addition, the specific angles of slope or tilt of the RF electrodes 42A-42D and 44A-44D may be altered to help to configure specific directions and specific angles with which the RF electrodes 42A-42D and 44A-44D target RF energy to tissues (layers) and/or tissue depths to configure and to control desired or required RF heating profiles. Further, the RF electrode 42A-42D and 44A-44D angles may be altered depending on the degree of flexibility of the volume of tissue 47 engaged within the cavity 46.

With further reference to FIG. 4, an illustrative example of operation of the treatment applicator 40 according to the invention is described with reference to a thermally assisted skin rejuvenation treatment for purposes of disclosing the invention. However, the invention is not limited to this treatment application and envisions any of a variety of skin, tissue, and organ treatments are possible.

The treatment applicator 40 is placed on the surface 23 of an area of skin 21 and a negative pressure is applied to the treatment applicator 40 that is sufficient to drawn a volume of skin tissue 47 within the treatment cavity 46 to a particular depth and to maintain such position of the tissue volume 47, such that, certain skin layers and depths, represented by Zone A and Zone B, are disposed between and at least substantially cover multiple tilted RF electrodes 42A-42D and 44A-44D. In this application, Zone B may represent the reticular layer of the skin dermis comprising dense connective tissue and thick collagen fibers, while Zone A may represent the papillary layer of the dermis that is closest to the skin epidermis and comprises loose connective tissue with fine collagen and elastin fibers and portions folded into ridges and papillae extending into the epidermis. One or more electronically coupled RF electrodes, e.g., 42C-44C and 42D-44D, deliver RF energy to Zone B in a specific direction(s) and at a specific angle(s) to target the reticular layer. In addition, these RF electrodes may deliver RF energy to target a particular location or depth within the reticular layer. The RF energy the RF electrodes provide would be configured with a particular intensity or fluence, or with intensities or fluences within a particular range, and delivered under a given pressure P_{X} for a particular length of time to produce the desired thermal impact within the reticular layer, including shrinkage of collagen fibers that produces a tightening effect in the skin. Subsequent or prior to treatment of Zone B, one or more electronically coupled RF electrodes, e.g., 42A-44A and 42B-44B, deliver RF energy to Zone A in a specific direction(s) and at a specific angle(s) to target the papillary layer. In addition, these RF electrodes may deliver RF energy to target a particular location or depth within the papillary layer. The RF energy the RF electrodes 42A-44A and 42B-44B provide would be configured with a particular intensity or fluence, or with intensities or fluences within a particular range, and delivered under a given pressure P_{X} for a particular length of time to produce the desired thermal impact of fibroblast stimulation and collagenesis within the papillary layer that results from heating/wounding and consequent healing of the papillary layer, which forms new collagen and elastin fibers. RF induced heating the papillary layer or Zone A has a wrinkle reduction effect, while RF induced heating the reticular layer of Zone B has a skin tightening effect. Multiple and different treatment responses are thereby produced within the volume of target tissue 47 that are tissue (layer) specific and tissue depth specific.

Additionally or alternatively, the treatment applicator 40 and the multiple tilted RF electrodes 42A-42D and 44A-44D may be arranged and electronically coupled and operated in accordance with parameters that provide fractional treatments to a given volume of tissue 47. For instance, the papillary layer of Zone A and the reticular layer of Zone B may be treated with the bipolar RF electrodes 42A-42D and 44A-44D as described above to fractionally target RF energy to the particular layers to produce fractional thermal responses and treatment effects within the papillary and reticular layers of the volume of target tissue 47 that are tissue (layer) specific and depth specific. The treatment applicator 40 according to the invention thereby permits customized fractional treatments to different tissues (layers) and to different tissue depths that produce multiple and different treatment effects within a given volume of target tissue 47.

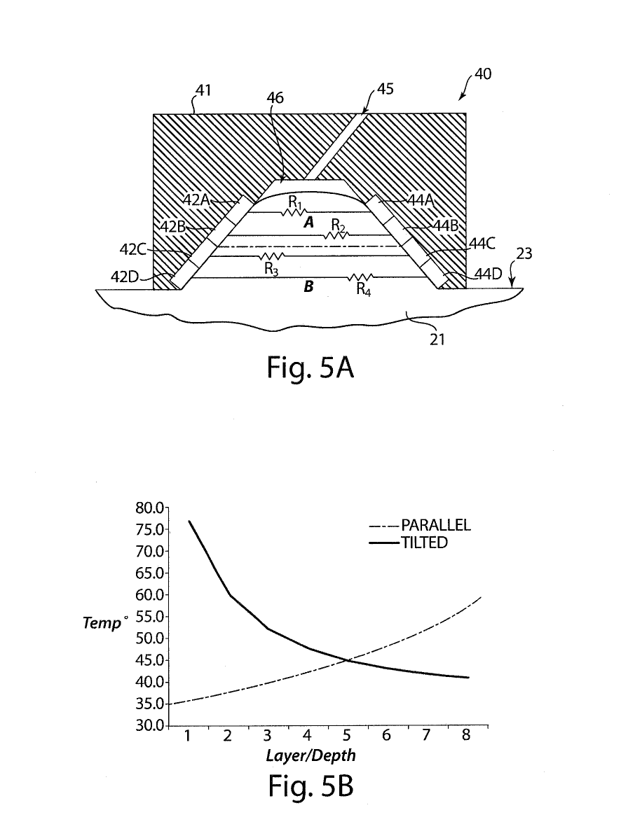

Referring to FIG. 5A and with further reference to FIG. 4, the amount of RF induced heating within a particular tissue type, layer and/or depth of the target tissue 47, such as within Zone A and within Zone B, not only may depend on the parameters described above to configure and target fractional RF energy to the target tissue 47, but may also depend on the resistivity R₁, R₂, R₃, and R₄ of the target tissue 47. As shown in FIG. 4, targeted tissue within Zone A may receive RF energy from two paired RF electrodes 42A, 44A and 42B, 44B, as described above, and a particular target tissue (layer) and/or depth of Zone A may have its own particular resistivity R₁ and R₂ to the applied RF energy. As a result, the target tissue (layer) and depth of Zone A would experience a consequent specific amount of RF induced heating and exhibit thermal responses related to its resistivity R₁ and R₂ to RF energy. Similarly, a particular target tissue (layer) and depth of Zone B would experience a consequent specific amount of RF induced heating and exhibit thermal responses related to its resistivity R₃ and R₄ to RF energy. The thermal responses within Zone and within Zone B may be different, as described in the skin rejuvenation example. The resistivity R₁, R₂ of Zone A and the resistivity R₃, R₄ of Zone B to RF energy illustrated in FIG. 7A would result when the multiple RF electrodes 42A-42D and 44A-44D are electronically coupled as described above and operate in a substantially parallel orientation to deliver RF energy between paired RF electrodes as indicated by arrows 48 shown in FIG. 4.

Tissues having relatively high impedance or resistivity Rx to RF energy, such as the reticular dermis and subcutaneous fat layers of skin, generate greater heat in response to RF current than tissue with relatively low resistivity and can account for thermal effects in deep tissue depths. Also, for instance, when coagulation is the desired treatment effect within a particular tissue depth, or a relatively intense or calm treatment effect is desired within deep or superficial tissues (layers) of the target tissue 47, the system 10 and 20, and/or the treatment applicator 40, may include the resistivity Rx of the specific tissues (layers) and/or specific depth as parameters for configuring and targeting RF energy. Use of tissue resistance, such as R₁, R₂ of Zone A and R₃, R₄ of Zone B, would help the system 10 and 20, and/or the treatment applicator 40 configure RF energy and target RF energy along the X, Z and/or Y-axes within Zone A and within Zone B to achieve the treatment impact desired, including, for instance, stimulation, coagulation, ablation and fragmentation. The treatment applicator 40 thereby customizes and further optimizes multiple and different fractional thermal responses and treatment effects within the given volume of target tissue 47.

Returning to the illustrative example of the skin rejuvenation treatment described with reference to FIG. 4, this treatment may be performed and completed with the treatment applicator 40 according to the invention in less time and with better results and quicker healing as a result of more precisely controlled heating profiles the treatment applicator 40 creates throughout Zone A and Zone B. For instance, in a single pass of the treatment applicator 40 shown in FIG. 4 over a given treatment area of skin 21, the RF electrodes 42A, 44A and 42B, 44B adjacent Zone A may operate alone or in conjunction with operation of the other RF electrodes 42C, 44C and 42D, 44D adjacent Zone B. The RF electrodes 42A, 44A and 42B, 44B adjacent Zone A may target and apply RF energy, e.g., configured relative to Zone A resistivity, at a lower intensity or fluence than the intensity or fluence of the RF energy, e.g., configured relative to Zone B resistivity, that the RF electrodes 42C, 44C and 42D, 44D may target and apply to Zone B, and for a longer duration than the duration of irradiation of Zone B. The longer duration of applying RF energy of a lower intensity to Zone A is required in order to stimulate fibroblasts and the slower process of collagenesis in the superficial papillary layer of Zone A tissue. The shorter duration of applying RF energy of a higher intensity to Zone B is required to deposit heat in Zone B tissue to wound and denature collagen fibers and thereby to shrink collagen, which is a rapid process, and to stimulate wound healing for formation of new collagen fibers around the denatured tissue. The RF electrodes 42A-42D and 44A-44D thereby selectively irradiate each of Zones A and B with customized fractional or nonfractional RF energy to achieve layered treatment to specific tissues (layers) and tissue depths. Such layered treatment of target tissue 47 using the treatment applicator 40 is appropriate for various treatments of the skin, as well as for other treatments of other soft tissue and organs that are comprised of different tissue types and tissue layers.

Referring to FIG. 5B, a graph illustrates comparative thermal profiles generated in irradiated target tissue 37 from use of parallel RF electrode pairs 32 and 34, such as shown in FIG. 3, and from use of the multiple tilted RF electrodes 42A-42D and 44A-44D of the treatment applicator 40 according to the invention, such as shown in FIG. 4, providing selective fractional RF treatment. The numbers 1 thru 8 indicated along the X-axis of the graph refer to the number of tissue layers affected with number 1 representing a tissue layer closest to the skin surface 23. As the graph of FIG. 7B illustrates, a wider range of RF energy-induced heating temperatures result from the most superficial layer 1 through the deepest layer 8 using multiple tilted RF paired electrodes 42A-42D and 44A-44D versus straight and substantially parallel RF electrodes 32 and 34. In addition, the graph illustrates that RF energy-induced heating occurs to greater depths within a given volume of tissue using pairs of tilted RF electrodes, suggesting the effects of customized RF energy depend on at least the resistivity R₁₋₄ of the targeted tissue layers and/or tissue depths.

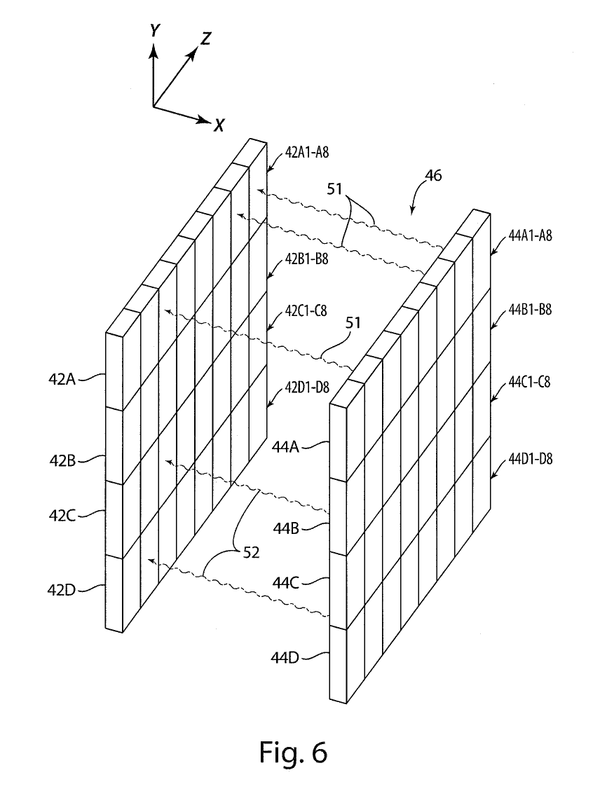

Referring to FIG. 6 and with further reference to FIG. 4, a perspective view of the multiple tilted bipolar RF electrodes 42A-42D and 44A-44D illustrates one potential configuration and arrangement, and operation, of the RF electrodes 42A-42D and 44A-44D of the treatment applicator 40 according to the invention that may provide fractional, and optionally nonfractional, RF treatments targeted to specific tissues (layers) and/or locations or depths within tissues (layers) of a given volume of target tissue 47. The illustrated configuration and arrangement shown in FIG. 6 may be disposed at any orientation within the treatment cavity 46, along any of the internal surfaces of the treatment application 40 that define the cavity 46. FIG. 6 also illustrates the capability and flexibility that the multiple RF electrodes 42A-42D and 44A-44D provide in configuring and customizing three-dimensional RF treatments to the target tissue volume 47 in order to produce the described multiple and different tissue-specific and depth-specific thermal responses and treatment effects. While FIG. 6 does not illustrate a specific angle of the slope or tilt of the RF electrodes 42A-42D and 44A-44D, it is understood that when the configuration and arrangement shown in FIG. 6 is positioned within the treatment applicator 40, the RF electrodes 42A-42D and 44A-44D are positioned at specific angles relative to the treatment cavity 46 and the volume of target tissue 47 engaged within the cavity 46, as shown in and described above with reference to FIG. 4, to deliver RF energy in specific directions and at specific angles to the target tissue 47.

In one configuration of the treatment applicator 40 according to the invention as shown in FIG. 6, one or more of the tilted RF electrodes 42A-42D and 44A-44D may be configured as fractionated RF electrodes that may fractionally target RF energy to particular layers and depths of the target tissue 47, and/or to particular locations or depths within given tissue layers. The RF electrodes 42A-42D may include fractionated electrodes 42A1-A8, B1-B8, C1-C8, and D1-D8 with each electrode having a given number of fractions, e.g., 8. The other RF electrodes 44A-44D may also include fractionated electrodes and 44A1-A5, B1-B8, C1-C8, and D1-D8 that may include the same number, e.g., 8, of fractions. Depending on the treatment application and the tissue layer and/or tissue depth targeted for treatment, one or more fractions of the fractionated electrode 42A1-A5, B1-B8, C1-C8, and D1-D8 may be electronically coupled or paired with one or more fractions of the fractionated electrode and 44A1-A5, 44 B1-B8, C1-C8, and D1-D8 to deliver RF energy in specific directions and at specific angles. For instance, the 42A1 fraction may be paired with the 44A1 fraction, the 42B1 fraction may be paired with the 44B1 fraction and so on. The paired fractions 42-44A1, 42-44B1, 42-44C1, 42-44D1, 42-44A2, 42-44B2, 42-44C2, 42-44D2, etc. convey RF current in a bipolar modality, as indicated by arrows 51 and 52 shown in FIG. 6, to provide RF energy in specific directions and at specific angles to target a particular tissue layer, tissue depth, and/or a particular location or depth with a given tissue layer when the target tissue 47 is positioned in contact with the fractionated RF electrodes. The paired fractions of the fractionated RF electrodes such as, for instance, the paired fractions 42A1-A8 and 44A1-A8, the paired fractions 42B1-B8 and 44B1-B8, the paired fractions 42C1-C8 and 44C1-C8, etc. may thereby deliver RF energy along an X-axis and along a Z-axis of the tissue volume 47, e.g., to target a particular tissue layer, and may also deliver RF energy along a Y-axis of the tissue volume 47, e.g., to target a particular depth within the tissue layer and/or within the tissue volume 47. In addition, the paired RF fractions may further deliver RF energy to target a particular location within a given tissue layer or at a given tissue depth. The fractionated RF electrodes can thereby deliver RF energy in specific directions and at specific angles along the X, Z and/or Y axes of the volume of target tissue 47 and create specific heating profiles, e.g., three-dimensional, and thermal responses within the tissue volume 47. Such selective and targeted RF treatment would thereby produce treatment effects that are tissue (layer) specific and depth specific.

Returning to the example of the skin rejuvenation application described above with reference to FIG. 4, the fractionated RF electrodes 42A1-A8 and 44A1-A8 may be paired and electrodes 42B1-B8 and 44B1-B8 may be paired to target fractional RF energy to Zone A of the target tissue 47 in order to selectively treat a particular tissue layer and/or tissue depth, and/or a particular location or depth within a given layer, of Zone A. Fractionated RF electrodes 42C1-C8 and 44C1-C8 may be paired and electrodes 42D1-D8 and 44D1-D8 may be paired to target fractional RF energy to Zone B of the target tissue 47. Similarly, a particular tissue layer and/or tissue depth, and/or a particular location or depth within a given layer, of Zone B may be selectively treated. The RF treatment the fractionated electrodes 42A1-A8; 44A1-A8 and 42B1-B8; 44B1-B8 target to Zone A may be different and have different effects from the RF treatment the fractionated electrodes 42C1-C8; 44C1-C8 and 42D1-D8; 44BD-D8 target to Zone B.

One of ordinary skill in the art can appreciate that RF current can be conveyed between one or more electronically coupled or paired individual fractions of the fractionated RF electrodes 42A1-A8, 42B1-B8, C1-C8, and D1-D8 and 44A1-A5, 44 B1-B8, C1-C8, and D1-D8, such that, RF energy is conveyed between paired individual fractions including, for instance, paired individual fractions 42A1-44A1, paired fractions 42A6-44A6, paired fractions 42C6-44C6, and paired fractions 42D7-44D7, as indicated by arrows 51 and 52 shown in FIG. 5, to fractionally target RF energy in different directions and at different angles. In this manner, fractional and different RF treatments are possible and are more precisely controlled along the X, Z and/or Y axes within a given volume of three-dimensional target tissue 47 using the treatment applicator 40 according to the invention. In addition, it is understood that in operation of the treatment head 40 according to the invention, electronic coupling or pairing of one or more fractionated RF electrodes 42A1-A8, B1-B8, C1-C8, D1-D8 and 44 B1-B8, C1-C8, D1-D8, and/or of one or more individual fractions 42A1, 42B1, 44A1, 44B1, etc. of each electrode, may be switched to electronically couple or pair with different fractionated RF electrodes and with different individual fractions. It is understood that the electronic coupling or pairing of RF electrodes, and/or of individual fractions of RF electrodes, may be switched to more precisely target RF energy, as illustrated and described below with reference to FIGS. 7A and 7B. This helps to provide more precise control of the distribution and depth of RF energy within the target tissue 47 and, as a result, more precise control of a thermal profile(s) produced within the volume of target tissue 47. In addition, the electronic couplings or pairings of fractionated RF electrodes, and/or of individual fractions, may be switched to alter specific directions and specific angles at which RF energy is applied, as well as may be switched in accordance with the configuration characteristics of RF energy, e.g., fluence, applied to a particular tissue layer, a particular tissue depth and/or a particular location or depth within a given tissue layer to achieve desired treatment effects.

With further reference to FIGS. 4 and 6, the system 10 and 20, and/or the treatment head 40, may operate one or more RF electrodes 42A-42D and 44A-44D independently, simultaneously, sequentially and/or in any order or pattern relative to the operation of the other RF electrodes. Such operation may depend on the required or desired treatment application and the specific directions and specific angles at which RF energy is targeted to the tissue volume 47. In addition, as described below with reference to FIGS. 7A and 7B, the system 10 and 20, and/or the treatment applicator 40, may operate one or more fractionated RF electrodes 42A1-A8, B1-B8, C1-C8, D1-D8 and 44 B1-B8, C1-C8, D1-D8, and/or one or more individual RF fractions 42A1, 42B1, 44A1, 44B1, etc. of given fractionated electrodes, independently, simultaneously, sequentially and/or in any order or pattern relative to the operation of other fractionated RF electrodes and of other individual RF fractions. In addition, as described, one or more RF electrodes 42A-42D and 44A-44D, or one or more fractionated RF electrodes 42A1-A8, B1-B8, C1-C8, D1-D8 and 44 B1-B8, C1-C8, D1-D8, and/or one or more individual RF fractions 42A1, 42B1, 44A1, 44B1, etc., may deliver RF energy configured with the same or different parameters and characteristics. In this respect, the configuration and arrangement of the multiple RF electrodes 42A-42D and 44A-44D of the treatment applicator 40 and their operation according to the invention provide flexibility and adaptability in delivering and targeting RF energy to specific tissues (layers) and/or to specific tissue depths in order to produce selective thermal responses and customized treatment effects that are tissue specific and/or depth specific.

For instance, paired RF electrodes 42A, 44A and 42B, 44B may operate independently, simultaneously, sequentially, or in a specific order or pattern to target fractional RF energy to a specific Zone A layer, a specific Zone A depth and/or a particular location or depth within a given Zone A layer, such that, RF energy applies to Zone A and Zone B remains unaffected. Alternatively, one or more electronically coupled or paired fractionated RF electrodes 42A1-A8, 44A1-A8, and 42B1-B8, 44B1-Bi, and/or paired individual RF fractions, e.g., 42A1, 42A2, 42B1, 42B2 and 44A1, 44A2, 44B1, 44B2, etc., may operate independently, simultaneously, sequentially or in a specific order or pattern to target fractional RF energy to the specific layer and/or depth of Zone A, and/or to a particular location or depth within a given layer of Zone A, to produce fractional heating and thermal responses within Zone A that are tissue (layer) specific and depth specific along the X, Z and/or Y axes of Zone A. The distribution and depth of RF energy and thereby RF induced heating is thereby controlled within Zone A along the X, Z and/or Y-axes while portions and areas of Zone A tissue may remain advantageously unaffected and intact. As mentioned, such intact areas or portions may serve a number of purposes that may relate to the type of treatment applied, the particular tissues (layers) treated, the particular depths treated, and/or the particular location or depth within a given layer treated. Such purposes may include, but are not limited to, helping to provide mechanical support to the treated tissue and/or the surrounding untreated tissue, or helping to maintain a blood supply to treated and damaged tissue to stimulate and accelerate healing and recovery. Zone B may be treated with similarly targeted fractional RF energy to produce a distribution and depth of RF energy and thereby RF induced heating within Zone B along the X, Z and/or Y-axes while portions and areas of Zone B tissue may remain advantageously unaffected and intact.

In addition, electronic coupling and operation of one or more RF electrodes 42A-42D and 44A-44D, or of one or more fractionated RF electrodes 42A1-A8, B1-B8, C1-C8, D1-D8 and 44 B1-B8, C1-C8, D1-D8 and/or individual RF fractions, e.g., 42A1, 42A2, 42B, 42B and 44A1, 44A2, 44B1, 44B2, etc., may depend on any of the parameters described above that configure fractional RF energy with certain characteristics, as well as any of the parameters and characteristics related to the type of treatment application and the skin, tissue and/or organ to which fractional RF treatment is applied.

Further, one or more RF electrodes 42A-42D and 44A-44D, or one or more fractionated RF electrodes 42A1-A8, B1-B8, C1-C8, D1-D8 and 44 B1-B8, C1-C8, D1-D8 and/or individual RF fractions, e.g., 42A1, 42A2, 42B, 42B and 44A1, 44A2, 44B1, 44B2, etc., may operate in a continuous mode to deliver continuous RF current, e.g., for a specific length of time, or, alternatively or additionally, may operate in a pulsed mode to deliver pulsed RF current with specific pulse duration, width, and frequency. These modes of operation, and the parameters of such modes, can depend on the type of treatment application the treatment applicator 40 provides, the skin, tissue and/or organ to which the applicator 40 applies fractional RF energy, and the RF induced heating required or desired along the X, Z, and/or Y-axes of the target tissue 47.

One or more RF electrodes 42A-42D and 44A-44D, or one or more fractionated RF electrodes 42A1-A8, B1-B8, C1-C8, D1-D8 and 44 B1-B8, C1-C8, D1-D8, may be positioned and/or spaced relative to other RF electrodes along any of the internal surfaces defining the treatment cavity 46 in order to help further pattern the specific directions and specific angles at which RF energy is delivered to target tissue 47 and to help further control the distribution and depth of RF energy within the target tissue 47.

Referring to FIGS. 7A and 7B, electronic coupling or pairing of the multiple tilted bipolar RF electrodes 42A-42D and 44A-44D, or electronic coupling or pairing of one or more fractionated RF electrodes 42A1-A8, B1-B8, C1-C8, D1-D8 and 44 B1-B8, C1-C8, D1-D8, and/or individual RF fractions, e.g., 42A1, 42A2, 42B, 42B and 44A1, 44A2, 44B1, 44B2, etc., may be switched before and/or during operation of the treatment head 40, such that, the RF current conveyed in a bipolar modality between paired RF electrodes, or between paired fractionated RF electrodes and/or paired individual RF fractions, flows through the treatment cavity 46 and through the volume of target tissue 47 in specific directions and at specific angles. For instance, as shown in FIG. 7A, the RF electrode 42A may be switched from a 42A-44A pairing, as shown in FIG. 6, to a 42A-44D pairing, such that, RF current flows between the RF electrodes 42A and 44D and through Zone A and Zone B in a specific direction and at a specific angle as shown by arrows 53A. Similarly the RF electrode 42D may be switched from a 42D-44D pairing, as shown in FIG. 6, to a 42D-44A pairing, such that, RF current flows between the RF electrodes 42D and 44A and through Zone A and Zone B in a specific direction and at a specific angle as shown by arrows 53B. The direction and the angle at which the electronically coupled RF electrodes 42A-44D target RF energy may be different from the direction and the angle at which the electronically coupled RF electrodes 42D-44A target RF energy. In addition, the RF energy delivered by the 42A-44D pairing and by the 42D-44A pairing may have different characteristics, e.g., fluence, such that, the RF electrodes 42A-44D and 42D-44A may produce different thermal impacts on the targeted Zone A and Zone B tissues (layers) and depths. FIGS. 7A and 7B illustrate the flexibility the treatment applicator 40 according to the invention provides in targeting RF energy in a pattern through a given volume of target tissue 47 and the capability of the treatment applicator 40 to thereby manipulate and control the distribution and depth of fractional, and nonfractional, RF energy and the resulting RF induced heating within the target tissue 47. The treatment applicator 40 according to the invention, therefore, produces controllable and predictable heating profiles and different and multiple desired thermal effects within Zone A and within Zone B.

In another instance, as shown in FIG. 7B, where the RF electrodes are fractionated electrodes, electronic coupling or pairing of one or more fractionated RF electrodes 42A1-A8, B1-B8, C1-C8, D1-D8 and 44 B1-B8, C1-C8, D1-D8 42A, and/or one or more individual RF fractions 42A1-A8, 42B1-B8, 44A1-A8, 44B1-B8, etc., may be switched before and/or during operation of the treatment head 40 to convey RF current in specific directions and at specific angles as shown by arrows 54A and 54B. FIG. 7B further illustrates the ability of the system 10 and 20, and/or the treatment applicator 40, for switching electronic coupling of one or more fractionated RF electrodes and of one or more individual RF fractions to alter the directions and the specific angles at which the treatment applicator 40 targets RF energy along the X, Z and/or Y axes of the volume of target tissue 47. This also illustrates the ability of the system 10 and 20, and/or the treatment applicator 40, to manipulate and precisely control the distribution and depth of fractional, and nonfractional, RF energy and the resulting RF induced heating within the target tissue 47. In this configuration, one or more individual RF fractions 42A1-A8 may electronically couple with one or more individual RF fractions 44D1-D8, and similarly one or more individual RF fractions 42D1-D8 may electronically couple with one or more individual RF fractions 44A1-A8, for precisely targeting of RF energy to a specific tissue (layer), a specific depth, and/or a specific location or depth within a given layer, within the target tissue 47.

As one of ordinary skill will appreciate and anticipate any RF electrodes 42A-42D may be switched to electronically couple with any other RF electrodes 44A-44D to target RF energy to different tissues (layers) and different tissue depths. In addition, one of ordinary skill will appreciate and anticipate any fractionated RF electrodes 42A1-A8, B1-B8, C1-C8, D1-D8 and 44 B1-B8, C1-C8, D1-D8, and/or any individual RF fractions, may be switched to facilitate relatively greater manipulation and more precise control of the specific directions and the specific angles with which the treatment applicator 40 targets RF energy to a particular tissue (layer), a particular tissue depth, and/or a particular location or depth within a given tissue layer, such that, the treatment applicator 40 produces reliable and consistent, and different, treatment effects. The invention is not limited to the arrangements of the RF electrodes shown in FIGS. 4 and 6 and FIGS. 7A and 7B, as well as the configuration and operation of electronically coupled RF electrodes, and envisions that any of a variety of arrangements of RF electrodes, and any configuration and operation of electronically coupled RF electrodes, is possible with the treatment applicator 40 according to the invention.

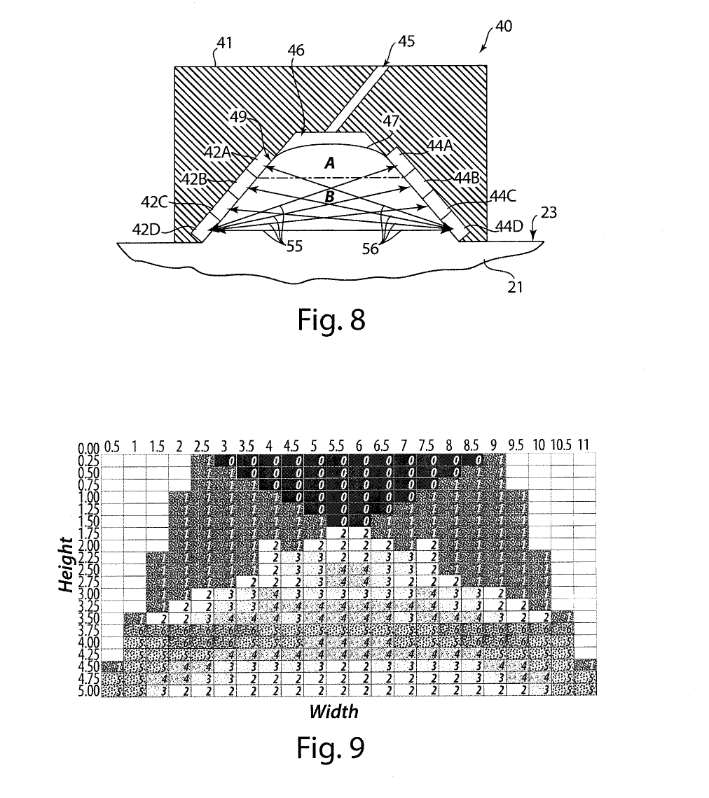

Referring to FIG. 8, another illustrative arrangement and configuration of electronically coupled multiple bipolar RF electrodes 42A-44D and 44A-44D RF is shown. The RF electrodes 42A-44D and 44A-44D shown in FIG. 8 may be programmed and/or operated in accordance with parameters set by the system 10 and 20, and/or the treatment applicator 40, to activate independently, simultaneously, sequentially and/or in a given order or pattern relative to operation of one or more of the other RF electrodes, as similarly described above with reference to FIGS. 4 and 6 and FIGS. 7A and 7B. In addition, one or more RF electrodes 42A-42D may be programmed and/or operated to conduct RF current with one or more other RF electrodes 44A-44D with which they are electronically coupled to target RF energy in one or more different directions and at one or more different angles.

For instance, one RF electrode 42D may be programmed and/or operated to activate alone or in conjunction with the other RF electrodes 42A-42C and to conduct RF current with each RF electrode 44A-44D with which it is electronically coupled to target RF energy in specific and different directions and at specific and different angles, as illustrated by arrows 55 shown in FIG. 8. In this configuration, the RF electrode 42D may be programmed and/or operated to activate to conduct RF current with each RF electrode 44A-44D in a sequential pattern, or to conduct RF current simultaneously with each RF 44A-44D. RF energy is thereby delivered in multiple and different directions and angles. As a result of the different directions and different angles with which the RF electrode 42D conducts current with the RF electrodes 44A-44D, RF energy may flow through different tissue layers and/or different depths of the target tissue 47 positioned within Zone and within Zone B. RF electrode 42D can thereby target fractional RF energy to Zone A and Zone B to provide fractional treatment, along X, Z, and/or Y axes, of the volume of target tissue 47 that produces tissue layer specific and/or tissue depth specific treatment effects.

Still referring to FIG. 8, in another instance, any of the RF electrodes 42A, 42B, 42C and 42D may be programmed and/or operated to activate to conduct RF current with the RF electrode 44D with which they are electronically coupled to target RF energy in specific and different directions and at specific and different angles, as illustrated by arrows 56 shown in FIG. 8. Each RF electrode 42A, 42B, 42C and 42D may conduct RF current with RF electrode 44D sequentially and/or simultaneously. As a result of the different directions and different angles with which the RF electrode 42A-42D conduct current with the RF electrode 44D, RF energy may flow through different tissue layers and/or different depths of the target tissue 47 positioned within Zone and within Zone B. RF electrodes 42A-42D can thereby target fractional RF energy to Zone A and Zone B to provide fractional treatment, along X, Z, and/or Y axes, of the volume of target tissue 47 that produces tissue layer specific and/or tissue depth specific treatment effects.

Similarly, one or more of the fractionated RF electrodes 42A1-A8, B1-B8, C1-C8, D1-D8 and 44 B1-B8, C1-C8, D1-D8, and/or one or more individual RF fractions, shown in FIGS. 6 and 7B, may be programmed and/or operated to activate at the same or different times relative to the other fractionated RF electrodes and/or other individual RF fractions to conduct RF current between electronically coupled RF electrodes and RF fractions as described above. Fractionated RF electrodes, and/or individual RF fractions, may activate independently, simultaneously, sequentially and in any order or pattern relative to other RF electrodes and individual RF fractions to deliver RF energy in different directions and at different angles to the volume of target tissue 47, such that, RF energy produces tissue layer specific and/or tissue depth specific treatment effects within Zone A and within Zone B.

In addition, the multiple RF electrodes 42A-42D and 44A-44D, or fractionated RF electrodes 42A1-A8, B1-B8, C1-C8, D1-D8 and 44 B1-B8, C1-C8, D1-D8, and/or one or more individual RF fractions, may activate, or may be operated by the system 10 and 20 and/or the treatment applicator 40, in a continuous mode to deliver continuous RF currents for a specific length of time, or, alternatively or additionally, may operate in a pulsed mode to deliver pulsed RF current with specific pulse duration, width, and frequency. These parameters would depend on the tissue treatment application and the heating profiles required or desired within the target tissue to produce customized tissue-specific and depth-specific treatment impacts.

Further, one or more of the RF electrodes 42A-42D and 44A-44D illustrated in FIGS. 4 and 6, FIGS. 7A and 7B, and FIG. 8 may include one or more bipolar RF micro-needle electrodes designed and constructed to precisely target and deliver RF energy directly into the volume of target tissue 47 and, more particularly, into a specific layer and/or depth of the target tissue 47. Bipolar RF micro-needle electrodes are designed for minimally invasive tissue treatment and are configured to confine a heating profile within the target tissue 47 between the bipolar needle pairs. Alternatively, one or more of the RF electrodes 42A-42D and 44A-44D may include an array of bipolar RF micro-needle electrodes. Like the bipolar RF electrodes 42A-42D and 44A-44D, the arrays of micro-needle electrodes may be selectively electronically coupled and programmed and/or operated to activate to conduct RF current as described above to target RF energy to specific tissue types, layers, and/or depths to thereby achieve multiple and different treatment effects, e.g., within Zone A and within Zone B.

Referring to FIG. 9, a chart illustrates a calculated model of the distribution and depth of RF induced heating of a given heating profile that is proportional to and corresponds with the pattern of RF current distribution flowing through a volume of target tissue 47, such as that shown in FIG. 8. The model assumes the volume of target tissue 47, e.g., engaged within the treatment cavity 46, has a height of about 5 mm at its highest point and a width of about 11 mm at its widest span. However, these dimensions are for illustrative purposes only and do not limit this aspect of the invention, wherein the invention anticipates that the volume of target tissue 47 can have any dimensions. The chart illustrates the distribution of heat through the treated tissue 47 along its width, the X-axis, and along its height, the Y-axis. Numerals 4, 5 and 6 indicate greater RF induced heating with 6 being the warmest temperatures of the ranges of temperatures represented by numerals 4, 5 and 6, and numerals 1, 2 and 3 indicate lower RF induced heating with 3 being the warmest temperatures of the ranges of temperatures represented by numerals 1, 2, and 3. Zero 0 indicates no heating. As the chart indicates RF induced heating relates to tissue depth with relatively deeper tissue having greater resistivity to RF energy and, therefore, experiencing higher RF induced temperatures. Fractional RF depth-specific thermal treatments thereby may be achieved with patterning of the directions and the angles of RF energy paths through specific targeted tissue types, layers and/or depths, as illustrated in FIG. 8.

Referring to FIGS. 10 and 11, in another aspect, the invention provides the treatment applicator 40 with a combination of different energy-emitting components to provide multi-modality treatment of skin and tissue conditions and pathologies. The treatment applicator 40 may include one or more electronically coupled, tilted bipolar RF electrodes 62 and 64, and/or may include one or more electronically coupled arrays of bipolar RF micro-needle electrodes 62 and 64. Alternatively, the treatment applicator 40 may include one or more paired fractionated RF electrodes 62A1-A8, B1-B8, C1-C8, D1-D8 and 64 B1-B8, C1-C8, D1-D8 similar to those shown in FIG. 6 and FIGS. 7A and 7B. In addition, the treatment applicator 40 further includes one or more ultrasound energy-emitting devices 72 and 74, e.g., ultrasound transducers, positioned within the treatment applicator 40 along one or more of the internal surfaces defining the treatment cavity 46. The one or more ultrasound emitting devices 72 and 74 deliver ultrasound energy to the cavity 46 and thereby conduct ultrasound energy through the target tissue 47. Alternative, or additionally, two or more ultrasound emitting devices 72 and 74 may be functionally coupled ultrasound transducers that conduct ultrasound energy between them and deliver ultrasound energy to the cavity 46 and thereby the volume of target tissue 47.

Generally, the treatment applicator 40 described thus far is disclosed in relation to the use of bipolar RF electrodes, and/or micro-needle electrodes 62 and 64, to produce fractional, or nonfractional, RF induced heating and treatment in target tissues. As shown in FIG. 11, fractionated RF electrodes 62A1-A8, B1-B8, C1-C8, D1-D8 and 64 B1-B8, C1-C8, D1-D8 may be electronically coupled and selectively switched, and may be activated, as described above with reference to FIG. 6 and FIGS. 7A and 7B to target RF energy to specific tissue (layers), specific tissue depths, and/or to a specific location or depth within a given tissue layer of the target tissue 47. In addition, individual RF fractions 62A1-A8, 64A1-A8, 62B1-B8, 64B1-B8, etc., may be electronically coupled paired and selectively switched, and may be activated, as described above with reference to FIG. 6 and FIGS. 7A and 7B.

For instance, in one application the treatment applicator 40 according to the invention may be configured to provide cellulite treatment and may employ one or more tilted bipolar RF electrodes or micro-needle electrodes 62A, 64A and 62B, 64B, and/or one or more fractionated electrodes 62A1-A8, 62B1-B8, 62BC1-C8, to deliver RF energy to the superficial skin layers, such as the papillary dermis that may be represented by Zone A shown in FIG. 10, to stimulate fibroblasts and direct collagenesis. With the one or more ultrasound emitting devices 72 and 74, the applicator 40 can alternatively and/or additionally deliver high and low intensity, focused or regular, ultrasound energy to deeper tissues, such as subcutaneous fat layers located below the dermal layers that may be represented by at least a portion of Zone B shown in FIG. 10, for purposes of cellular and extra cellular matrix (ECM) destruction, which may be required or desired for cellulite treatment. While the ultrasound energy helps to destroy the underlying cellular infrastructure and matrix related to cellulite, the fractional RF energy delivered to the more superficial layers, e.g., papillary layer, may help to smooth out cellulite dimples. The treatment applicator 40 according to the invention can thereby deliver two different energy modalities with a single unit and thereby achieve multiple and different treatment effects depending on the specific layers and/or the specific depths to which RF energy and ultrasound energy are applied, e.g., in specific directions and at specific angles, to the target tissue 40.

The one or more ultrasound emitting devices 72 and 74 are configured to deliver ultrasound energy to a specific target tissue, such as tissue that creates a mechanical disturbance and destruction in response to ultrasound energy that causes cell death and destruction of the ECM. Such ultrasound energy may pass through overlying tissue as it flows to a tightly-focused, specific tissue within the volume of target tissue 47. In certain treatment applications, the rate of ultrasound energy deposition in the target tissue 47 may exceed the rate of heat dissipation, such that, a rapid rise in temperature in the target tissue 47 may be achieved. As a result, thermal ablation can be produced with deposition of ultrasound energy that creates local cavitations or formation of microchannels in the target tissue 47 with little heating of adjacent tissues. Irreversible cell death occurs in areas of cavitations or microchannels and such areas of tissue necrosis are typically sharply defined. Accurately targeting tissue with high-intensity, focused ultrasound energy allows the treatment applicator 40 according to the invention to precisely ablate a specific tissue, layer and/or depth within the target tissue 47, such as, for instance, a specific subcutaneous layer at a specific depth, without affecting or damaging surrounding tissue.

The treatment applicator 40 shown in FIG. 10 can thereby target RF energy and ultrasound energy to specific tissue layers along the X, Z and/or Y axes of the target tissue 47 to achieve multiple and different treatment effects that are tissue layer specific and depth specific.

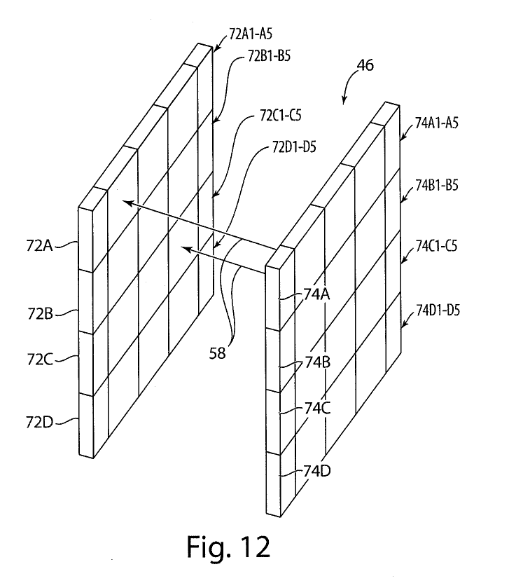

Referring to FIG. 12 and with further reference to FIG. 10, the one or more ultrasound emitting devices 72 and 74 may include ultrasound transducers configured to function independently without coupling with other transducers. Alternatively, or additionally, the one or more ultrasound emitting devices 72 and 74 include, as mentioned, at least two functionally coupled ultrasound transducers 72 and 74. Further, the one or more ultrasound emitting devices 72 and 74 may include one or more fractionated ultrasonic transducers including multiple subcomponents or fractions 72A1-A8, B1-B8, C1-C8, D1-D8 and 74 B1-B8, C1-C8, D1-D8, e.g., phase array transducers. The fractionated ultrasonic transducers 72A1-A8, B1-B8, C1-C8, D1-D8 and 74 B1-B8, C1-C8, D1-D8 may be configured to operate independently, e.g., without functional coupling, or may be configured to functionally couple for operation.

The system 10 and 20, and/or the treatment applicator 40, may be programmed and/or operate to activate one or more ultrasound emitting devices 72 and 74, and/or one or more individual transducer fractions 72A1-A8, B1-B8, C1-C8, D1-D8 and 74 B1-B8, C1-C8, D1-D8 within a given fractionated transducer, independently, simultaneously, sequentially, and/or in any given order or pattern, e.g., with respect to activation of other transducers and other individual transducer fractions. In addition, operation and activation of each ultrasound emitting device 72 and 74, or one or more transducer fractions 72A1-A8, B1-B8, C1-C8, D1-D8 and 74 B1-B8, C1-C8, D1-D8 of the fractionated transducers, may occur relative to operation or activation of one or more of the multiple bipolar RF electrodes or micro-needle electrodes 42A-42D and 44A-44D, and fractionated RF electrodes described above.

As shown in FIG. 12, in one configuration of the treatment applicator 40 according to the invention, the applicator 40 includes one or more fractionated ultrasound transducers 72A1-A8, B1-B8, C1-C8, D1-D8 and 74 B1-B8, C1-C8, D1-D8 72 and 74, with each fractionated transducer including a given number of transducer fractions. Ultrasound transducer fractions 72A1-A5, 72B1-B5, 74A1-A5, 7B1-B5, etc. may be configured to function independently to deliver ultrasound energy in a specific direction and at a specific angle to the volume of target tissue 47. Alternatively or additionally ultrasound transducer fractions 72A1-A5, 72B1-B5, 74A1-A5, 7B1-B5, etc. may be functionally coupled in order to deliver ultrasound energy in a specific direction and at a specific angle, as illustrated by arrows 58 in FIG. 12, to a particular tissue (layer), a particular tissue depth, and/or a particular location or depth within a given tissue layer of the volume of target tissue 47. In addition, one or more transducer fractions 72A1-A5, 72B1-B5, 74A1-A5, 7B1-B5, etc., may be configured to deliver different ultrasound waves having different characteristics. Different configurations of ultrasound energy may thereby treat a particular tissue (layer), a particular tissue depth, and/or a particular location or depth within a given tissue layer of the target tissue 47. The treatment applicator 40 thereby applies ultrasound energy in a precisely controlled configuration along the X-axis, and/or along the Z-axis, to target specific tissue layers and/or specific locations or depths within a given tissue layer. The treatment applicator 40 thereby also applies ultrasound energy in a precisely controlled configuration along the Y-axis to specific tissue depths and/or locations within the volume of target tissue 47. The treatment application 40 according to the invention thereby can achieve different and multiple desired or required ultrasound treatment effects with a given volume of target tissue 47.

It is understood that while the treatment applicator 40 shown in FIG. 10 may be configured with ultrasound emitting devices 72 and 74 and/or with multiple fractionated ultrasound-emitting devices, e.g., transducers, 72A1-A8, B1-B8, C1-C8, D1-D8 and 74 B1-B8, C1-C8, D1-D8 72, in addition to being configured with multiple RF electrodes or micro-needles 62 and 64 and/or multiple fractionated RF electrodes 62A1-A8, B1-B8, C1-C8, D1-D8 and 64 B1-B8, C1-C8, D1-D8, the invention anticipates the treatment applicator 40 according to the invention may be configured solely with the ultrasound emitting devices 72 and 74 and/or with the fractionated ultrasound-emitting devices, e.g., transducers, 72A1-A8, B1-B8, C1-C8, D1-D8 and 74 B1-B8, C1-C8, D1-D8 72 to provide ultrasound energy for tissue specific and/or tissue depth specific ultrasound treatment to the volume of target tissue 47.

Any of the treatments described above employing the treatment applicator 40 according to the invention and operating in the RF energy modality only, or in combination with the ultrasound energy modality, may employ mechanical manipulation devices and/or techniques in order to facilitate determination of the particular tissue (layer), the particular tissue depth, and/or the particular location or depth within a given tissue layer, within the target tissue 40 to receive energy treatment. The treatments described above using the treatment applicator 40 according to the invention may also employ pre- and/or post-treatment of the target tissue 47, as well as other concomitant modalities or treatments to help to facilitate treatment of the target tissue 47, and/or to increase the safety of treatment, and/or to help to increase the precise susceptibility of the target tissue 47 to a particular treatment energy. For instance, the treatment applicator 40 may be employed to target RF energy and/or ultrasound energy before or after specific cooling of the target tissue 47. The treatment applicator 40 may also be used with a monitoring device and/or techniques to assist with location of the particular tissue (layer), the particular tissue depth, and/or the particular location or depth within a given tissue layer, to be treated or being treated. Such monitoring device and/or techniques may be used to monitor the treatment impact during and/or after treatment energy is applied. Such monitoring devices and/or techniques may include using the same technologies to apply treatment energy, such as, for instance, ultrasound and radiofrequency modalities to measure and monitor the ultrasound wave velocity within the target tissue 47 during and after treatment, and to measure temperature and/or impedance and conductivity of the target tissue 47.

Having thus described at least one illustrative aspect of the invention, various alterations, modifications, and improvements will readily occur to those skilled in the art. Such alterations, modifications, and improvements are intended to be within the scope and spirit of the invention. Accordingly, the foregoing description is by way of example only and is not intended as limiting. The invention's limit is defined only in the following claims and the equivalents thereto.

What is claimed is:

1. A system for fractional treatment of a condition or pathology of a tissue, the system comprising:
a treatment applicator disposed and configured to deliver treatment energy to a target tissue, the treatment applicator defining within its interior a treatment cavity to engage the target tissue;
multiple bipolar RF electrodes disposed within the treatment applicator and along predetermined internal surfaces that define the treatment cavity, each bipolar electrode being disposed at an angle relative to the treatment cavity and being configured to generate RF energy;
the multiple bipolar RF electrodes including a first set of at least two bipolar RF electrodes disposed along at least two internal surfaces of the treatment cavity and being electronically coupled to operate in a bipolar modality to deliver RF energy to the treatment cavity in a specific direction and at a specific angle so that the at least two bipolar RF electrodes selectively target RF energy to at least one of: a specific layer, a specific depth, and a specific location or depth within a specific layer of a first zone within the target tissue; and
an RF energy source operatively coupled to the multiple bipolar RF electrodes.

2. The system of claim 1 wherein the first set of bipolar RF electrodes targets RF energy configured in accordance with one or more parameters to treat the first zone within the target tissue.

3. The system of claim 2 including a second set of at least two bipolar RF electrodes disposed along at least two internal surfaces of the treatment cavity and being electronically coupled to operate in a bipolar modality to deliver RF energy to the treatment cavity in a specific direction and at a specific angle so that the at least two bipolar RF electrodes of the second set selectively target RF energy to at least one of: a specific layer, a specific depth, and a specific location or depth within a specific layer of a second zone within the target tissue;

4. The system of claim 3 wherein the second set of bipolar RF electrodes targets RF energy configured in accordance with one or more parameters to treat the second zone within the target tissue.

5. The system of claim 3 wherein the first set of bipolar RF electrodes produces a treatment effect in the first zone different from the treatment effect the second set of bipolar RF electrodes produces in the second zone.

6. The system of claim 4 wherein RF energy the first set of bipolar RF electrodes targets to the first zone is different from RF energy the second set of bipolar RF electrodes targets to the second zone.

7. The system of claim 6 wherein the first set of bipolar RF electrodes produces a treatment effect in the first zone different from the treatment effect the second set of bipolar RF electrodes produces in the second zone.

8. The system of claim 5 wherein the treatment effects in the first zone and in the second zone of the target tissue are fractional treatment effects.

9. The system of claim 6 wherein the treatment effects in the first zone and in the second zone of the target tissue are fractional treatment effects.

10. The system of claim 3 wherein the at least two bipolar RF electrodes of the first set are disposed in a transverse orientation relative to one another on opposite surfaces of the treatment cavity.

11. The system of claim 10 wherein the at least two bipolar RF electrodes of the second set are disposed in a transverse orientation relative to one another on opposite surfaces of the treatment cavity.

12. The system of claim 11 wherein one of more of the multiple bipolar RF electrodes are configured so that electronic coupling of the bipolar RF electrodes can switch, wherein electronic coupling of one of the bipolar RF electrodes of the first set can switch to electronically couple with one of the bipolar RF electrodes of the second set to change the specific direction and the specific angle of RF energy to the treatment cavity.

13. The system of claim 1 including each bipolar RF electrode of the multiple bipolar RF electrodes disposed at the angle relative to the treatment cavity to facilitate contact between the bipolar RF electrode and the target tissue.

14. The system of claim 5 wherein the first set of bipolar RF electrodes delivers RF energy to the treatment cavity in the specific direction and at the specific angle to target RF energy along an X axis and along a Z axis of at least one of: the specific layer, the specific depth, and the specific location or depth of the first zone within the target tissue.

15. The system of claim 14 wherein the first set of bipolar RF electrodes delivers RF energy to the treatment chamber in the specific direction and at the specific angle to target RF energy along an along a Y axis of at least one of: the specific layer, the specific depth, and the specific location or depth of the first zone within the target tissue.

16. The system of claim 15 wherein RF energy the first set of bipolar RF electrodes targets to the first zone produces an RF induced heating profile within the first zone.

17. The system of claim 16 wherein the second set of bipolar RF electrodes delivers RF energy to the treatment chamber in the specific direction and at the specific angle to target RF energy along an X axis and along a Z axis of at least one of: the specific layer, the specific depth, and the specific location or depth of the second zone within the target tissue.

18. The system of claim 17 wherein the second set of bipolar RF electrodes delivers RF energy to the treatment chamber in the specific direction and at the specific angle to target RF energy along an along a Y axis of at least one of: the specific layer, the specific depth, and the specific location or depth of the second zone within the target tissue.

19. The system of claim 18 wherein RF energy the second set of bipolar RF electrodes targets to the second zone produces an RF induced heating profile within the second zone.

20. The system of claim 19 wherein the RF induced heating profile within the first zone produces one or more treatment effects different from the one or more treatment effects the RF induced heating profile within the second zone produces.

21. The system of claim 1 including at least two ultrasound transducers disposed within the treatment applicator and along predetermined internal surfaces that define the treatment cavity, each ultrasound transducer being disposed at an angle relative to the treatment cavity;
the ultrasound transducers further disposed and configured to deliver ultrasound energy in a specific direction and at a specific angle to target ultrasound energy to at least one of: a specific layer, a specific depth, and a specific location or depth within a specific layer within the target tissue; and
an ultrasound energy source operatively coupled to the ultrasound transducers.

22. The system of claim 21 wherein the ultrasound transducers produce one or more treatment effects within at least one of: the specific layer, the specific depth, and the specific location or depth within the specific layer within the target tissue.

23. The system of claim 22 wherein one or more treatment effects the ultrasound transducers produce are different from the treatment effects the first set of bipolar electrodes produces in the first zone and are different from the treatment effects the second set of bipolar electrodes produces in the second zone.

24. The system of claim 21 including a PC and a microprocessor configured to operate the first and the second sets of paired bipolar RF electrodes and the ultrasound transducers.

25. The system of claim 24 wherein the PC and the microprocessor operate the first and the second sets of paired bipolar RF electrodes and the ultrasound transducers in at least one of: a continuous mode and a pulsed mode.

26. The system of claim 1 wherein the first set of at least two opposing bipolar RF electrodes includes at least two fractionated bipolar RF electrodes including one or more RF fractions.

27. A treatment applicator for providing fractional treatment of a condition or pathology of a tissue, the treatment applicator comprising:
a treatment cavity defined within an interior of the treatment applicator and configured to engage a three-dimensional volume of target tissue;
multiple bipolar RF electrodes disposed along internal surfaces defining the treatment cavity, each bipolar RF electrode being disposed at an angle relative to the treatment cavity;
an RF energy source operatively coupled to the multiple bipolar RF electrodes.
at least two bipolar RF electrodes disposed along at least two internal surfaces of the treatment cavity and being electronically coupled to operate in a bipolar modality to deliver RF energy to the treatment cavity in a specific direction and at a specific angle so that the paired bipolar RF electrodes selectively target RF energy to at least one of: a specific layer, a specific depth, and a specific location or depth within a specific layer to produce one or more different RF treatment effects within the volume of target tissue.

28. The treatment applicator of claim 27 further including at least two ultrasound transducers disposed along at least two internal surfaces of the treatment cavity and being configured to deliver ultrasound energy to the treatment cavity in a specific direction and at a specific angle to target ultrasound energy to at least one of: a specific layer, a specific depth, and a specific location or depth within a specific layer to produce one or more different ultrasound treatment effects within the volume of target tissue.

29. A system for fractional treatment of a condition or pathology of a tissue, the system comprising:
a treatment applicator disposed and configured to deliver treatment energy to a target tissue, the treatment applicator defining within its interior a treatment cavity to engage the target tissue;
multiple ultrasound emitting devices disposed within the treatment applicator and along predetermined internal surfaces that define the treatment cavity, each ultrasound emitting device being disposed at an angle relative to the treatment cavity and being configured to generate ultrasound energy;
the multiple ultrasound emitting devices including a first set of at least two ultrasound emitting devices disposed along at least two internal surfaces of the treatment cavity and being further disposed to deliver ultrasound energy to the treatment cavity in a specific direction and at a specific angle so that the at least two ultrasound emitting devices of the first set selectively target ultrasound energy to at least one of: a specific layer, a specific depth, and a specific location or depth within a specific layer of a first zone within the target tissue; and
an ultrasound energy source operatively coupled to the multiple ultrasound emitting devices.

30. The system of claim 29, including the multiple ultrasound emitting devices including a second set of at least two ultrasound emitting devices disposed along at least two internal surfaces of the treatment cavity and being further disposed to deliver ultrasound energy to the treatment cavity in a specific direction and at a specific angle so that the at least two ultrasound emitting devices of the second set selectively target ultrasound energy to at least one of: a specific layer, a specific depth, and a specific location or depth within a specific layer of a second zone within the target tissue.
